(19) United States
(12) Patent Application Publication
(10) Pub. No.: US 2023/0277569 A1
(43) Pub. Date: Sep. 7, 2023

(54) Compositions and Methods for Treating, Ameliorating and/or Preventing Cancer and Kits for the Same

(71) Applicant: THE WISTAR INSTITUTE, Philadelphia, PA (US)

(72) Inventor: Dario C. Altieri, Philadelphia, PA (US)

(21) Appl. No.: 18/115,945

(22) Filed: Mar. 1, 2023

Related U.S. Application Data
(60) Provisional application No. 63/315,871, filed on Mar. 2, 2022.

(51) Int. Cl. A61K 31/675; A61P 35/00; A61K 47/20; A61K 47/26; A61K 47/24

(52) U.S. Cl. CPC A61K 31/675; A61P 35/00; A61K 47/20; A61K 47/26; A61K 47/24

(57) ABSTRACT

Described herein is a method of treating, ameliorating and/or preventing cancer in a subject in need thereof. The method includes administering to the subject therapeutically effective amounts of Gamitrinib according to one of certain specific administration schemes (protocols). Also described herein is a kit for performing the methods of the disclosure.

### CROSS-REFERENCE TO RELATED APPLICATIONS

This application claims priority under 35 U.S.C. § 119(e) to U.S. Provisional Patent Application No. 63/315,871, filed Mar. 2, 2022. The entire content of this application is hereby incorporated by reference herein.

### STATEMENT REGARDING FEDERALLY SPONSORED RESEARCH

This invention was made with government support under CA225913 awarded by the National Institutes of Health, and W81XWH-13-11-0193 and W81XWH-18-1-0334 awarded by the United States Department of Defense. The government has certain rights in the invention.

### BACKGROUND

Gamitrinib (GA mitochondrial matrix inhibitor) is a first-in-class mitochondrial-targeted inhibitor of organelle protein folding. Gamitrinib has been shown to be effective as an antitumor agent (see e.g., U.S. Pat. No. 8,466,140). However, dosages, formulations and routes of administration of Gamitrinib suitable for treating, ameliorating and/or preventing cancer in subjects are currently unknown.

Therefore, there is a need to identify novel compositions and/or administration protocols relating to Gamitrinib for treating, ameliorating, and/or preventing cancer. The present invention addresses this need.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1C depict the effects of Gamitrinib on ion channel activities in accordance with some embodiments. FIG. 1A: Gamitrinib (10 μM) or relevant control was incubated with the individual channel-containing samples and the % inhibition of conductance compared to control was quantified. Mean±SD. *, p=0.02; ***, p<0.0001. FIG. 1B: Recording of hERG currents in the presence of control or the indicated increasing concentrations of Gamitrinib. Representative experiment. FIG. 1C: HEK293 cells were transfected with hERG and analyzed for inhibition of hERG currents in the presence of increasing concentrations of Gamitrinib or control terfenadine. The IC₅₀ values for each compound tested are indicated.

FIGS. 2A-2C depict certain aspects of Gamitrinib PK in rats, in accordance with some embodiments. FIG. 2A: Gamitrinib (5 mg/kg) was injected IV in Sprague-Dawley rats and blood samples collected at the indicated time intervals were analyzed for Gamitrinib concentrations (Cₘₐₓ). Data from three individual animals and t_{1/2} values (mean±SD) are shown. FIG. 2B: The conditions are as in FIG. 2A and plasma samples from rats administered IV Gamitrinib were analyzed for Gamitrinib or 17-AG concentrations. LLOQ, lower limit of quantification. FIG. 2C: Male and female Sprague-Daley rats administered Gamitrinib IV at three dose levels (1, 10 and 25 mg/kg/dose) twice weekly were analyzed for Gamitrinib concentrations (Cₘₐₓ) on d 1, 4 and 29 of the dosing phase (mean±SD). LLOQ, lower limit of quantification.

FIGS. 3A-3C depict certain aspects of Gamitrinib toxicity in rats, in accordance with some embodiments. FIG. 3A-3B: Males and females Sprague-Dawley rats were administered IV Gamitrinib at the indicated dose levels of 1, 10 and 25 mg/kg twice weekly for 29 d (dosing phase) and body weight values or blood samples collected at the end of the dosing phase were analyzed for the indicated clinical-chemistry parameters (mean±SD). *, p=0.01-0.03; **, p=0.001-0.002; ***, p=0.0004-<0.0001; ns, not significant. FIG. 3C: Kidney histology of study rats (animal numbers in parentheses) administered vehicle or Gamitrinib IV (25 mg/kg/dose). Vehicle (#0001), normal kidney; Vehicle (#0004), minimal kidney degeneration/regeneration; Gamitrinib (#0306), slight kidney tubule degeneration/regeneration; Gamitrinib (#304), marked kidney tubule degeneration/regeneration. Representative images. Scale bar, 300 μm.

FIGS. 4A-4B depict certain aspects of Gamitrinib toxicity in dogs, in accordance with some embodiments. In FIGS. 4A-4B, Male and female beagle dogs were administered IV Gamitrinib at the indicated dose levels of 1.25, 3.33 and 6.25 mg/kg twice weekly for 36 d and blood samples collected prior to the initiation of dosing (predose) and on d 36 of the dosing phase (dosing) were analyzed for the indicated clinical-chemistry parameters (mean±SD). *, p=0.01-0.04; ***, p=0.001; ns, not significant.

FIGS. 5A-5B show certain aspects of the synthesis of Gamitrinib, in accordance with some embodiments. FIG. 5A: Stepwise chemical synthesis of Gamitrinib linking the Hsp90 ATPase inhibitor 17-AAG to the mitochondrial import carrier, triphenylphosphonium via a hexylamine linker. FIG. 5B: Workflow of Gamitrinib drug product with preparation of a GMP Gamitrinib Injectable Suspension (GIS, particle size, <200 nm) by microfluidization.

FIG. 6 depicts certain aspects of cytochrome (CYP) inhibition, in accordance with some embodiments. Increasing concentrations of Gamitrinib or relevant control were incubated with the indicated CYP isoforms and analyzed for % inhibition. The individual IC₅₀ values of Gamitrinib or control are indicated per each CYP tested.

FIG. 7 depicts the electrophysiologic studies, in accordance with some embodiments. Male and female beagle dogs were administered IV Gamitrinib at the indicated dose levels of 1.25, 3.33 and 6.25 mg/kg twice weekly and electrocardiograms (ECGs) were obtained from unanesthetized animals once during the predose phase (predose) and 1 to 2 h post end of infusion on d 32 of the dosing phase (dosing). Mean±SD.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The studies as described herein include a first study directed to a preclinical characterization of Gamitrinib, and a second study directed to a phase I clinical trial of Gamitrinib. In the first study, the toxicity of Gamitrinib was determined in vitro as well as in two exemplary animal models (Sprague-Dawley rats and beagle dogs), and the pharmacokinetics of Gamitrinib were studied using Sprague-Dawley rats as a model. In the second study, a phase I clinical trial to determine the safety profile of Gamitrinib in human subjects is carried out.

Accordingly, in some aspects, the present invention is directed to a method of treating, ameliorating and/or preventing cancer in a subject by administering to the subject Gamitrinib according to an administration scheme (protocol). In some aspects, the present invention is directed to a kit for carrying out the method.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the instant specification along with a compound and/or composition that may also treat or prevent a disease or disorder contemplated herein. In some embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the instant specification with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the instant specification within or to the patient such that it may perform its intended function. Each carrier must be “acceptable” in the sense of being compatible with the other ingredients of the formulation, including the compound useful within the instant specification, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the instant specification, and are physiologically acceptable to the patient. The “pharmaceutically acceptable carrier” may further include a pharmaceutically acceptable salt of the compound useful within the instant specification. Other additional ingredients that may be included in the pharmaceutical compositions used in the practice of the instant specification are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In some embodiments, the subject is human.

As used herein, the term “treatment” or “treating” is defined as the application or administration of a therapeutic agent, i.e., a compound useful within the instant specification (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

As used herein, the term “Gamitrinib” refers to 17-Demethoxy-17-((6-(triphenylphosphonio)hexyl)amino)geldanamycin or (6-(((4E,6Z,8S,9S,10E,12S,13R,14S,16R)-9-(carbamoyloxy)-13-hydroxy-8,14-dimethoxy-4,10,12,16-tetramethyl-3,20,22-trioxo-2-azabicyclo[16.3.1]docosa-1(21),4,6,10,18-pentaen-19-yl)amino)hexyl)triphenylphosphonium, which is illustrated as the following structure:

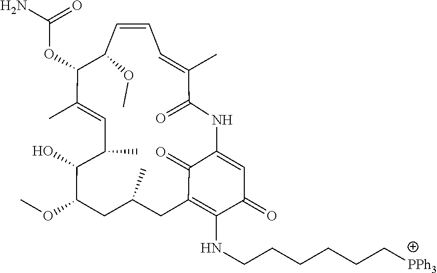

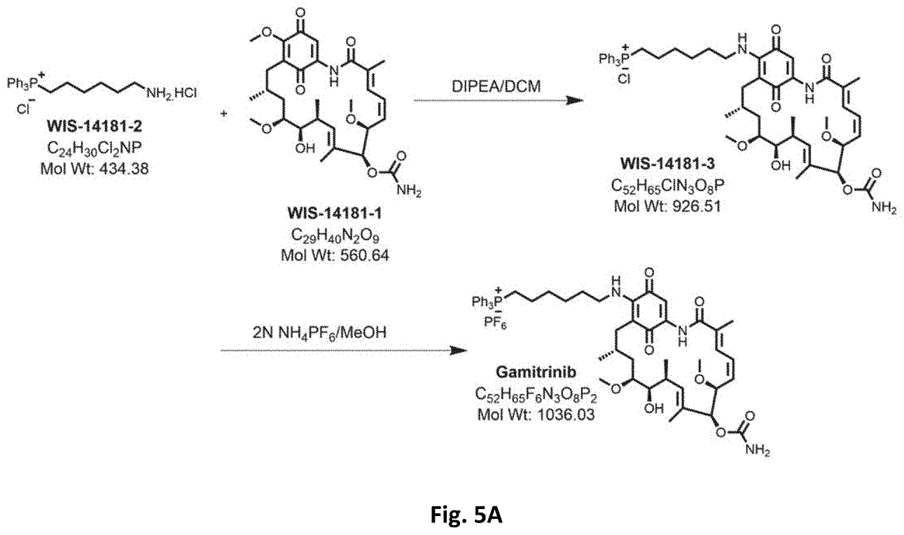

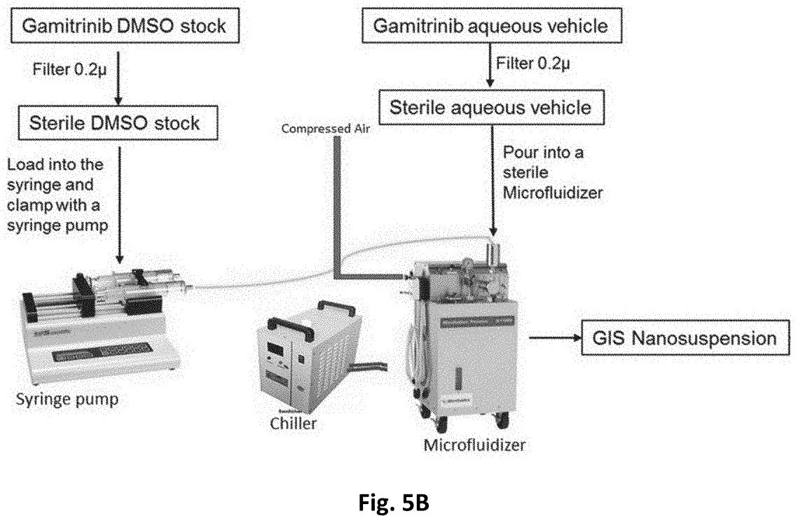

or a salt, solvate, isotopically labelled derivative, stereoisomer, tautomer, or geometric isomer thereof, and any mixtures thereof. The term “Gamitrinib” as used herein includes the hexafluorophosphate salt as shown, for example, in FIG. 5A herein.

### Compositions & Methods

In some aspects, the present invention is directed to compositions that can be used within a method of treating, ameliorating and/or preventing cancer in a subject in need thereof, as well as methods of manufacturing such compositions.

The present disclosure provides a pharmaceutical formulation suitable for parenteral formulation, the formulation comprising Gamitrinib and at least one, two, three, four, five, or all six of the following: water, DMSO, polysorbate 80, lecithin (Lipoid S100), sucrose, and dextrose.

In certain embodiments, the present disclosure provides a method of generating a Gamitrinib pharmaceutical formulation suitable for parenteral formulation, the method comprising subjecting to microfluidization a mixture of a DMSO-containing solution of Gamitrinib and a water-containing solution of polysorbate 80, lecithin (Lipoid S100), sucrose, and dextrose.

In certain embodiments, the pharmaceutical formulation comprises Gamitrinib, water, DMSO, polysorbate 80, lecithin (Lipoid S100), sucrose, and dextrose.

In certain embodiments, the pharmaceutical formulation comprises about 1-25 mg/mL Gamitrinib. In certain embodiments, the pharmaceutical formulation comprises about 1 mg/mL Gamitrinib. In certain embodiments, the pharmaceutical formulation comprises about 2 mg/mL Gamitrinib. In certain embodiments, the pharmaceutical formulation comprises about 3 mg/mL Gamitrinib. In certain embodiments, the pharmaceutical formulation comprises about 4 mg/mL Gamitrinib. In certain embodiments, the pharmaceutical formulation comprises about 5 mg/mL Gamitrinib. In certain embodiments, the pharmaceutical formulation comprises about 6 mg/mL Gamitrinib. In certain embodiments, the pharmaceutical formulation comprises about 7 mg/mL Gamitrinib. In certain embodiments, the pharmaceutical formulation comprises about 8 mg/mL Gamitrinib. In certain embodiments, the pharmaceutical formulation comprises about 9 mg/mL Gamitrinib. In certain embodiments, the pharmaceutical formulation comprises about 10 mg/mL Gamitrinib. In certain embodiments, the pharmaceutical formulation comprises about 11 mg/mL Gamitrinib. In certain embodiments, the pharmaceutical formulation comprises about 12 mg/mL Gamitrinib. In certain embodiments, the pharmaceutical formulation comprises about 13 mg/mL Gamitrinib. In certain embodiments, the pharmaceutical formulation comprises about 14 mg/mL Gamitrinib. In certain embodiments, the pharmaceutical formulation comprises about 15 mg/mL Gamitrinib. In certain embodiments, the pharmaceutical formulation comprises about 16 mg/mL Gamitrinib. In certain embodiments, the pharmaceutical formulation comprises about 17 mg/mL Gamitrinib. In certain embodiments, the pharmaceutical formulation comprises about 18 mg/mL Gamitrinib. In certain embodiments, the pharmaceutical formulation comprises about 19 mg/mL Gamitrinib. In certain embodiments, the pharmaceutical formulation comprises about 20 mg/mL Gamitrinib. In certain embodiments, the pharmaceutical formulation comprises about 21 mg/mL Gamitrinib. In certain embodiments, the pharmaceutical formulation comprises about 22 mg/mL Gamitrinib. In certain embodiments, the pharmaceutical formulation comprises about 23 mg/mL Gamitrinib. In certain embodiments, the pharmaceutical formulation comprises about 24 mg/mL Gamitrinib. In certain embodiments, the pharmaceutical formulation comprises about 25 mg/mL Gamitrinib.

In certain embodiments, the pharmaceutical formulation comprises equal to or greater than about 1 mg/mL Gamitrinib. In certain embodiments, the pharmaceutical formulation comprises equal to or greater than about 2 mg/mL Gamitrinib. In certain embodiments, the pharmaceutical formulation comprises equal to or greater than about 3 mg/mL Gamitrinib. In certain embodiments, the pharmaceutical formulation comprises equal to or greater than about 4 mg/mL Gamitrinib. In certain embodiments, the pharmaceutical formulation comprises equal to or greater than about 5 mg/mL Gamitrinib. In certain embodiments, the pharmaceutical formulation comprises equal to or greater than about 6 mg/mL Gamitrinib. In certain embodiments, the pharmaceutical formulation comprises equal to or greater than about 7 mg/mL Gamitrinib. In certain embodiments, the pharmaceutical formulation comprises equal to or greater than about 8 mg/mL Gamitrinib. In certain embodiments, the pharmaceutical formulation comprises equal to or greater than about 9 mg/mL Gamitrinib. In certain embodiments, the pharmaceutical formulation comprises equal to or greater than about 10 mg/mL Gamitrinib. In certain embodiments, the pharmaceutical formulation comprises equal to or greater than about 11 mg/mL Gamitrinib. In certain embodiments, the pharmaceutical formulation comprises equal to or greater than about 12 mg/mL Gamitrinib. In certain embodiments, the pharmaceutical formulation comprises equal to or greater than about 13 mg/mL Gamitrinib. In certain embodiments, the pharmaceutical formulation comprises equal to or greater than about 14 mg/mL Gamitrinib. In certain embodiments, the pharmaceutical formulation comprises equal to or greater than about 15 mg/mL Gamitrinib. In certain embodiments, the pharmaceutical formulation comprises equal to or greater than about 16 mg/mL Gamitrinib. In certain embodiments, the pharmaceutical formulation comprises equal to or greater than about 17 mg/mL Gamitrinib. In certain embodiments, the pharmaceutical formulation comprises equal to or greater than about 18 mg/mL Gamitrinib. In certain embodiments, the pharmaceutical formulation comprises equal to or greater than about 19 mg/mL Gamitrinib. In certain embodiments, the pharmaceutical formulation comprises equal to or greater than about 20 mg/mL Gamitrinib. In certain embodiments, the pharmaceutical formulation comprises equal to or greater than about 21 mg/mL Gamitrinib. In certain embodiments, the pharmaceutical formulation comprises equal to or greater than about 22 mg/mL Gamitrinib. In certain embodiments, the pharmaceutical formulation comprises equal to or greater than about 23 mg/mL Gamitrinib. In certain embodiments, the pharmaceutical formulation comprises equal to or greater than about 24 mg/mL Gamitrinib. In certain embodiments, the pharmaceutical formulation comprises equal to or greater than about 25 mg/mL Gamitrinib.

In certain embodiments, the pharmaceutical formulation comprises equal to or less than about 1 mg/mL Gamitrinib. In certain embodiments, the pharmaceutical formulation comprises equal to or less than about 2 mg/mL Gamitrinib. In certain embodiments, the pharmaceutical formulation comprises equal to or less than about 3 mg/mL Gamitrinib. In certain embodiments, the pharmaceutical formulation comprises equal to or less than about 4 mg/mL Gamitrinib. In certain embodiments, the pharmaceutical formulation comprises equal to or less than about 5 mg/mL Gamitrinib. In certain embodiments, the pharmaceutical formulation comprises equal to or less than about 6 mg/mL Gamitrinib. In certain embodiments, the pharmaceutical formulation comprises equal to or less than about 7 mg/mL Gamitrinib. In certain embodiments, the pharmaceutical formulation comprises equal to or less than about 8 mg/mL Gamitrinib. In certain embodiments, the pharmaceutical formulation comprises equal to or less than about 9 mg/mL Gamitrinib. In certain embodiments, the pharmaceutical formulation comprises equal to or less than about 10 mg/mL Gamitrinib. In certain embodiments, the pharmaceutical formulation comprises equal to or less than about 11 mg/mL Gamitrinib. In certain embodiments, the pharmaceutical formulation comprises equal to or less than about 12 mg/mL Gamitrinib. In certain embodiments, the pharmaceutical formulation comprises equal to or less than about 13 mg/mL Gamitrinib. In certain embodiments, the pharmaceutical formulation comprises equal to or less than about 14 mg/mL Gamitrinib. In certain embodiments, the pharmaceutical formulation comprises equal to or less than about 15 mg/mL Gamitrinib. In certain embodiments, the pharmaceutical formulation comprises equal to or less than about 16 mg/mL Gamitrinib. In certain embodiments, the pharmaceutical formulation comprises equal to or less than about 17 mg/mL Gamitrinib. In certain embodiments, the pharmaceutical formulation comprises equal to or less than about 18 mg/mL Gamitrinib. In certain embodiments, the pharmaceutical formulation comprises equal to or less than about 19 mg/mL Gamitrinib. In certain embodiments, the pharmaceutical formulation comprises equal to or less than about 20 mg/mL Gamitrinib. In certain embodiments, the pharmaceutical formulation comprises equal to or less than about 21 mg/mL Gamitrinib. In certain embodiments, the pharmaceutical formulation comprises equal to or less than about 22 mg/mL Gamitrinib. In certain embodiments, the pharmaceutical formulation comprises equal to or less than about 23 mg/mL Gamitrinib. In certain embodiments, the pharmaceutical formulation comprises equal to or less than about 24 mg/mL Gamitrinib. In certain embodiments, the pharmaceutical formulation comprises equal to or less than about 25 mg/mL Gamitrinib.

In certain embodiments, the pharmaceutical formulation comprises about 0.5-15% DMSO. In certain embodiments, the pharmaceutical formulation comprises about 0.5% DMSO. In certain embodiments, the pharmaceutical formulation comprises about 1% DMSO. In certain embodiments, the pharmaceutical formulation comprises about 1.5% DMSO. In certain embodiments, the pharmaceutical formulation comprises about 2% DMSO. In certain embodiments, the pharmaceutical formulation comprises about 2.5% DMSO. In certain embodiments, the pharmaceutical formulation comprises about 3% DMSO. In certain embodiments, the pharmaceutical formulation comprises about 3.5% DMSO. In certain embodiments, the pharmaceutical formulation comprises about 4% DMSO. In certain embodiments, the pharmaceutical formulation comprises about 4.5% DMSO. In certain embodiments, the pharmaceutical formulation comprises about 5% DMSO. In certain embodiments, the pharmaceutical formulation comprises about 5.5% DMSO. In certain embodiments, the pharmaceutical formulation comprises about 6% DMSO. In certain embodiments, the pharmaceutical formulation comprises about 6.5% DMSO. In certain embodiments, the pharmaceutical formulation comprises about 7% DMSO. In certain embodiments, the pharmaceutical formulation comprises about 7.5% DMSO. In certain embodiments, the pharmaceutical formulation comprises about 8% DMSO. In certain embodiments, the pharmaceutical formulation comprises about 8.5% DMSO. In certain embodiments, the pharmaceutical formulation comprises about 9% DMSO. In certain embodiments, the pharmaceutical formulation comprises about 9.5% DMSO. In certain embodiments, the pharmaceutical formulation comprises about 10% DMSO. In certain embodiments, the pharmaceutical formulation comprises about 10.5% DMSO. In certain embodiments, the pharmaceutical formulation comprises about 11% DMSO. In certain embodiments, the pharmaceutical formulation comprises about 11.5% DMSO. In certain embodiments, the pharmaceutical formulation comprises about 12% DMSO. In certain embodiments, the pharmaceutical formulation comprises about 12.5% DMSO. In certain embodiments, the pharmaceutical formulation comprises about 13% DMSO. In certain embodiments, the pharmaceutical formulation comprises about 13.5% DMSO. In certain embodiments, the pharmaceutical formulation comprises about 14% DMSO. In certain embodiments, the pharmaceutical formulation comprises about 14.5% DMSO. In certain embodiments, the pharmaceutical formulation comprises about 15% DMSO.

In certain embodiments, the pharmaceutical formulation comprises equal to or greater than about 0.5% DMSO. In certain embodiments, the pharmaceutical formulation comprises equal to or greater than about 1% DMSO. In certain embodiments, the pharmaceutical formulation comprises equal to or greater than about 1.5% DMSO. In certain embodiments, the pharmaceutical formulation comprises equal to or greater than about 2% DMSO. In certain embodiments, the pharmaceutical formulation comprises equal to or greater than about 2.5% DMSO. In certain embodiments, the pharmaceutical formulation comprises equal to or greater than about 3% DMSO. In certain embodiments, the pharmaceutical formulation comprises equal to or greater than about 3.5% DMSO. In certain embodiments, the pharmaceutical formulation comprises equal to or greater than about 4% DMSO. In certain embodiments, the pharmaceutical formulation comprises equal to or greater than about 4.5% DMSO. In certain embodiments, the pharmaceutical formulation comprises equal to or greater than about 5% DMSO. In certain embodiments, the pharmaceutical formulation comprises equal to or greater than about 5.5% DMSO. In certain embodiments, the pharmaceutical formulation comprises equal to or greater than about 6% DMSO. In certain embodiments, the pharmaceutical formulation comprises equal to or greater than about 6.5% DMSO. In certain embodiments, the pharmaceutical formulation comprises equal to or greater than about 7% DMSO. In certain embodiments, the pharmaceutical formulation comprises equal to or greater than about 7.5% DMSO. In certain embodiments, the pharmaceutical formulation comprises equal to or greater than about 8% DMSO. In certain embodiments, the pharmaceutical formulation comprises equal to or greater than about 8.5% DMSO. In certain embodiments, the pharmaceutical formulation comprises equal to or greater than about 9% DMSO. In certain embodiments, the pharmaceutical formulation comprises equal to or greater than about 9.5% DMSO. In certain embodiments, the pharmaceutical formulation comprises equal to or greater than about 10% DMSO. In certain embodiments, the pharmaceutical formulation comprises equal to or greater than about 10.5% DMSO. In certain embodiments, the pharmaceutical formulation comprises equal to or greater than about 11% DMSO. In certain embodiments, the pharmaceutical formulation comprises equal to or greater than about 11.5% DMSO. In certain embodiments, the pharmaceutical formulation comprises equal to or greater than about 12% DMSO. In certain embodiments, the pharmaceutical formulation comprises equal to or greater than about 12.5% DMSO. In certain embodiments, the pharmaceutical formulation comprises equal to or greater than about 13% DMSO. In certain embodiments, the pharmaceutical formulation comprises equal to or greater than about 13.5% DMSO. In certain embodiments, the pharmaceutical formulation comprises equal to or greater than about 14% DMSO. In certain embodiments, the pharmaceutical formulation comprises equal to or greater than about 14.5% DMSO. In certain embodiments, the pharmaceutical formulation comprises equal to or greater than about 15% DMSO.

In certain embodiments, the pharmaceutical formulation comprises equal to or less than about 0.5% DMSO. In certain embodiments, the pharmaceutical formulation comprises equal to or less than about 1% DMSO. In certain embodiments, the pharmaceutical formulation comprises equal to or less than about 1.5% DMSO. In certain embodiments, the pharmaceutical formulation comprises equal to or less than about 2% DMSO. In certain embodiments, the pharmaceutical formulation comprises equal to or less than about 2.5% DMSO. In certain embodiments, the pharmaceutical formulation comprises equal to or less than about 3% DMSO. In certain embodiments, the pharmaceutical formulation comprises equal to or less than about 3.5% DMSO. In certain embodiments, the pharmaceutical formulation comprises equal to or less than about 4% DMSO. In certain embodiments, the pharmaceutical formulation comprises equal to or less than about 4.5% DMSO. In certain embodiments, the pharmaceutical formulation comprises equal to or less than about 5% DMSO. In certain embodiments, the pharmaceutical formulation comprises equal to or less than about 5.5% DMSO. In certain embodiments, the pharmaceutical formulation comprises equal to or less than about 6% DMSO. In certain embodiments, the pharmaceutical formulation comprises equal to or less than about 6.5% DMSO. In certain embodiments, the pharmaceutical formulation comprises equal to or less than about 7% DMSO. In certain embodiments, the pharmaceutical formulation comprises equal to or less than about 7.5% DMSO. In certain embodiments, the pharmaceutical formulation comprises equal to or less than about 8% DMSO. In certain embodiments, the pharmaceutical formulation comprises equal to or less than about 8.5% DMSO. In certain embodiments, the pharmaceutical formulation comprises equal to or less than about 9% DMSO. In certain embodiments, the pharmaceutical formulation comprises equal to or less than about 9.5% DMSO. In certain embodiments, the pharmaceutical formulation comprises equal to or less than about 10% DMSO. In certain embodiments, the pharmaceutical formulation comprises equal to or less than about 10.5% DMSO. In certain embodiments, the pharmaceutical formulation comprises equal to or less than about 11% DMSO. In certain embodiments, the pharmaceutical formulation comprises equal to or less than about 11.5% DMSO. In certain embodiments, the pharmaceutical formulation comprises equal to or less than about 12% DMSO. In certain embodiments, the pharmaceutical formulation comprises equal to or less than about 12.5% DMSO. In certain embodiments, the pharmaceutical formulation comprises equal to or less than about 13% DMSO. In certain embodiments, the pharmaceutical formulation comprises equal to or less than about 13.5% DMSO. In certain embodiments, the pharmaceutical formulation comprises equal to or less than about 14% DMSO. In certain embodiments, the pharmaceutical formulation comprises equal to or less than about 14.5% DMSO. In certain embodiments, the pharmaceutical formulation comprises equal to or less than about 15% DMSO.

In certain embodiments, the pharmaceutical formulation comprises about 0.025-1% polysorbate 80. In certain embodiments, the pharmaceutical formulation comprises about 0.025% polysorbate 80. In certain embodiments, the pharmaceutical formulation comprises about 0.05% polysorbate 80. In certain embodiments, the pharmaceutical formulation comprises about 0.075% polysorbate 80. In certain embodiments, the pharmaceutical formulation comprises about 0.1% polysorbate 80. In certain embodiments, the pharmaceutical formulation comprises about 0.2% polysorbate 80. In certain embodiments, the pharmaceutical formulation comprises about 0.3% polysorbate 80. In certain embodiments, the pharmaceutical formulation comprises about 0.4% polysorbate 80. In certain embodiments, the pharmaceutical formulation comprises about 0.5% polysorbate 80. In certain embodiments, the pharmaceutical formulation comprises about 0.6% polysorbate 80. In certain embodiments, the pharmaceutical formulation comprises about 0.7% polysorbate 80. In certain embodiments, the pharmaceutical formulation comprises about 0.8% polysorbate 80. In certain embodiments, the pharmaceutical formulation comprises about 0.9% polysorbate 80. In certain embodiments, the pharmaceutical formulation comprises about 1% polysorbate 80.

In certain embodiments, the pharmaceutical formulation comprises equal to or greater than about 0.025% polysorbate 80. In certain embodiments, the pharmaceutical formulation comprises equal to or greater than about 0.05% polysorbate 80. In certain embodiments, the pharmaceutical formulation comprises equal to or greater than about 0.075% polysorbate 80. In certain embodiments, the pharmaceutical formulation comprises equal to or greater than about 0.1% polysorbate 80. In certain embodiments, the pharmaceutical formulation comprises equal to or greater than about 0.2% polysorbate 80. In certain embodiments, the pharmaceutical formulation comprises equal to or greater than about 0.3% polysorbate 80. In certain embodiments, the pharmaceutical formulation comprises equal to or greater than about 0.4% polysorbate 80. In certain embodiments, the pharmaceutical formulation comprises equal to or greater than about 0.5% polysorbate 80. In certain embodiments, the pharmaceutical formulation comprises equal to or greater than about 0.6% polysorbate 80. In certain embodiments, the pharmaceutical formulation comprises equal to or greater than about 0.7% polysorbate 80. In certain embodiments, the pharmaceutical formulation comprises equal to or greater than about 0.8% polysorbate 80. In certain embodiments, the pharmaceutical formulation comprises equal to or greater than about 0.9% polysorbate 80. In certain embodiments, the pharmaceutical formulation comprises equal to or greater than about 1% polysorbate 80.

In certain embodiments, the pharmaceutical formulation comprises equal to or less than about 0.025% polysorbate 80. In certain embodiments, the pharmaceutical formulation comprises equal to or less than about 0.05% polysorbate 80. In certain embodiments, the pharmaceutical formulation comprises equal to or less than about 0.075% polysorbate 80. In certain embodiments, the pharmaceutical formulation comprises equal to or less than about 0.1% polysorbate 80. In certain embodiments, the pharmaceutical formulation comprises equal to or less than about 0.2% polysorbate 80. In certain embodiments, the pharmaceutical formulation comprises equal to or less than about 0.3% polysorbate 80. In certain embodiments, the pharmaceutical formulation comprises equal to or less than about 0.4% polysorbate 80. In certain embodiments, the pharmaceutical formulation comprises equal to or less than about 0.5% polysorbate 80. In certain embodiments, the pharmaceutical formulation comprises equal to or less than about 0.6% polysorbate 80. In certain embodiments, the pharmaceutical formulation comprises equal to or less than about 0.7% polysorbate 80. In certain embodiments, the pharmaceutical formulation comprises equal to or less than about 0.8% polysorbate 80. In certain embodiments, the pharmaceutical formulation comprises equal to or less than about 0.9% polysorbate 80. In certain embodiments, the pharmaceutical formulation comprises equal to or less than about 1% polysorbate 80.

In certain embodiments, the pharmaceutical formulation comprises about 0.01-1% lecithin. In certain embodiments, the pharmaceutical formulation comprises about 0.01% lecithin. In certain embodiments, the pharmaceutical formulation comprises about 0.02% lecithin. In certain embodiments, the pharmaceutical formulation comprises about 0.03% lecithin. In certain embodiments, the pharmaceutical formulation comprises about 0.04% lecithin. In certain embodiments, the pharmaceutical formulation comprises about 0.05% lecithin. In certain embodiments, the pharmaceutical formulation comprises about 0.06% lecithin. In certain embodiments, the pharmaceutical formulation comprises about 0.07% lecithin. In certain embodiments, the pharmaceutical formulation comprises about 0.08% lecithin. In certain embodiments, the pharmaceutical formulation comprises about 0.09% lecithin. In certain embodiments, the pharmaceutical formulation comprises about 0.1% lecithin. In certain embodiments, the pharmaceutical formulation comprises about 0.2% lecithin. In certain embodiments, the pharmaceutical formulation comprises about 0.3% lecithin. In certain embodiments, the pharmaceutical formulation comprises about 0.4% lecithin. In certain embodiments, the pharmaceutical formulation comprises about 0.5% lecithin. In certain embodiments, the pharmaceutical formulation comprises about 0.6% lecithin. In certain embodiments, the pharmaceutical formulation comprises about 0.7% lecithin. In certain embodiments, the pharmaceutical formulation comprises about 0.8% lecithin. In certain embodiments, the pharmaceutical formulation comprises about 0.9% lecithin. In certain embodiments, the pharmaceutical formulation comprises about 1% lecithin.

In certain embodiments, the pharmaceutical formulation comprises equal to or greater than about 0.01% lecithin. In certain embodiments, the pharmaceutical formulation comprises equal to or greater than about 0.02% lecithin. In certain embodiments, the pharmaceutical formulation comprises equal to or greater than about 0.03% lecithin. In certain embodiments, the pharmaceutical formulation comprises equal to or greater than about 0.04% lecithin. In certain embodiments, the pharmaceutical formulation comprises equal to or greater than about 0.05% lecithin. In certain embodiments, the pharmaceutical formulation comprises equal to or greater than about 0.06% lecithin. In certain embodiments, the pharmaceutical formulation comprises equal to or greater than about 0.07% lecithin. In certain embodiments, the pharmaceutical formulation comprises equal to or greater than about 0.08% lecithin. In certain embodiments, the pharmaceutical formulation comprises equal to or greater than about 0.09% lecithin. In certain embodiments, the pharmaceutical formulation comprises equal to or greater than about 0.1% lecithin. In certain embodiments, the pharmaceutical formulation comprises equal to or greater than about 0.2% lecithin. In certain embodiments, the pharmaceutical formulation comprises equal to or greater than about 0.3% lecithin. In certain embodiments, the pharmaceutical formulation comprises equal to or greater than about 0.4% lecithin. In certain embodiments, the pharmaceutical formulation comprises equal to or greater than about 0.5% lecithin. In certain embodiments, the pharmaceutical formulation comprises equal to or greater than about 0.6% lecithin. In certain embodiments, the pharmaceutical formulation comprises equal to or greater than about 0.7% lecithin. In certain embodiments, the pharmaceutical formulation comprises equal to or greater than about 0.8% lecithin. In certain embodiments, the pharmaceutical formulation comprises equal to or greater than about 0.9% lecithin. In certain embodiments, the pharmaceutical formulation comprises equal to or greater than about 1% lecithin.

In certain embodiments, the pharmaceutical formulation comprises equal to or less than about 0.01% lecithin. In certain embodiments, the pharmaceutical formulation comprises equal to or less than about 0.02% lecithin. In certain embodiments, the pharmaceutical formulation comprises equal to or less than about 0.03% lecithin. In certain embodiments, the pharmaceutical formulation comprises equal to or less than about 0.04% lecithin. In certain embodiments, the pharmaceutical formulation comprises equal to or less than about 0.05% lecithin. In certain embodiments, the pharmaceutical formulation comprises equal to or less than about 0.06% lecithin. In certain embodiments, the pharmaceutical formulation comprises equal to or less than about 0.07% lecithin. In certain embodiments, the pharmaceutical formulation comprises equal to or less than about 0.08% lecithin. In certain embodiments, the pharmaceutical formulation comprises equal to or less than about 0.09% lecithin. In certain embodiments, the pharmaceutical formulation comprises equal to or less than about 0.1% lecithin. In certain embodiments, the pharmaceutical formulation comprises equal to or less than about 0.2% lecithin. In certain embodiments, the pharmaceutical formulation comprises equal to or less than about 0.3% lecithin. In certain embodiments, the pharmaceutical formulation comprises equal to or less than about 0.4% lecithin. In certain embodiments, the pharmaceutical formulation comprises equal to or less than about 0.5% lecithin. In certain embodiments, the pharmaceutical formulation comprises equal to or less than about 0.6% lecithin. In certain embodiments, the pharmaceutical formulation comprises equal to or less than about 0.7% lecithin. In certain embodiments, the pharmaceutical formulation comprises equal to or less than about 0.8% lecithin. In certain embodiments, the pharmaceutical formulation comprises equal to or less than about 0.9% lecithin. In certain embodiments, the pharmaceutical formulation comprises equal to or less than about 1% lecithin.

In certain embodiments, the pharmaceutical formulation comprises about 0.5-10% sucrose. In certain embodiments, the pharmaceutical formulation comprises about 0.5% sucrose. In certain embodiments, the pharmaceutical formulation comprises about 1% sucrose. In certain embodiments, the pharmaceutical formulation comprises about 1.5% sucrose. In certain embodiments, the pharmaceutical formulation comprises about 2% sucrose. In certain embodiments, the pharmaceutical formulation comprises about 2.5% sucrose. In certain embodiments, the pharmaceutical formulation comprises about 3% sucrose. In certain embodiments, the pharmaceutical formulation comprises about 3.5% sucrose. In certain embodiments, the pharmaceutical formulation comprises about 4% sucrose. In certain embodiments, the pharmaceutical formulation comprises about 4.5% sucrose. In certain embodiments, the pharmaceutical formulation comprises about 5% sucrose. In certain embodiments, the pharmaceutical formulation comprises about 5.5% sucrose. In certain embodiments, the pharmaceutical formulation comprises about 6% sucrose. In certain embodiments, the pharmaceutical formulation comprises about 6.5% sucrose. In certain embodiments, the pharmaceutical formulation comprises about 7% sucrose. In certain embodiments, the pharmaceutical formulation comprises about 7.5% sucrose. In certain embodiments, the pharmaceutical formulation comprises about 8% sucrose. In certain embodiments, the pharmaceutical formulation comprises about 8.5% sucrose. In certain embodiments, the pharmaceutical formulation comprises about 9% sucrose. In certain embodiments, the pharmaceutical formulation comprises about 9.5% sucrose. In certain embodiments, the pharmaceutical formulation comprises about 10% sucrose.

In certain embodiments, the pharmaceutical formulation comprises equal to or greater than about 0.5% sucrose. In certain embodiments, the pharmaceutical formulation comprises equal to or greater than about 1% sucrose. In certain embodiments, the pharmaceutical formulation comprises equal to or greater than about 1.5% sucrose. In certain embodiments, the pharmaceutical formulation comprises equal to or greater than about 2% sucrose. In certain embodiments, the pharmaceutical formulation comprises equal to or greater than about 2.5% sucrose. In certain embodiments, the pharmaceutical formulation comprises equal to or greater than about 3% sucrose. In certain embodiments, the pharmaceutical formulation comprises equal to or greater than about 3.5% sucrose. In certain embodiments, the pharmaceutical formulation comprises equal to or greater than about 4% sucrose. In certain embodiments, the pharmaceutical formulation comprises equal to or greater than about 4.5% sucrose. In certain embodiments, the pharmaceutical formulation comprises equal to or greater than about 5% sucrose. In certain embodiments, the pharmaceutical formulation comprises equal to or greater than about 5.5% sucrose. In certain embodiments, the pharmaceutical formulation comprises equal to or greater than about 6% sucrose. In certain embodiments, the pharmaceutical formulation comprises equal to or greater than about 6.5% sucrose. In certain embodiments, the pharmaceutical formulation comprises equal to or greater than about 7% sucrose. In certain embodiments, the pharmaceutical formulation comprises equal to or greater than about 7.5% sucrose. In certain embodiments, the pharmaceutical formulation comprises equal to or greater than about 8% sucrose. In certain embodiments, the pharmaceutical formulation comprises equal to or greater than about 8.5% sucrose. In certain embodiments, the pharmaceutical formulation comprises equal to or greater than about 9% sucrose. In certain embodiments, the pharmaceutical formulation comprises equal to or greater than about 9.5% sucrose. In certain embodiments, the pharmaceutical formulation comprises equal to or greater than about 10% sucrose.

In certain embodiments, the pharmaceutical formulation comprises equal to or less than about 0.5% sucrose. In certain embodiments, the pharmaceutical formulation comprises equal to or less than about 1% sucrose. In certain embodiments, the pharmaceutical formulation comprises equal to or less than about 1.5% sucrose. In certain embodiments, the pharmaceutical formulation comprises equal to or less than about 2% sucrose. In certain embodiments, the pharmaceutical formulation comprises equal to or less than about 2.5% sucrose. In certain embodiments, the pharmaceutical formulation comprises equal to or less than about 3% sucrose. In certain embodiments, the pharmaceutical formulation comprises equal to or less than about 3.5% sucrose. In certain embodiments, the pharmaceutical formulation comprises equal to or less than about 4% sucrose. In certain embodiments, the pharmaceutical formulation comprises equal to or less than about 4.5% sucrose. In certain embodiments, the pharmaceutical formulation comprises equal to or less than about 5% sucrose. In certain embodiments, the pharmaceutical formulation comprises equal to or less than about 5.5% sucrose. In certain embodiments, the pharmaceutical formulation comprises equal to or less than about 6% sucrose. In certain embodiments, the pharmaceutical formulation comprises equal to or less than about 6.5% sucrose. In certain embodiments, the pharmaceutical formulation comprises equal to or less than about 7% sucrose. In certain embodiments, the pharmaceutical formulation comprises equal to or less than about 7.5% sucrose. In certain embodiments, the pharmaceutical formulation comprises equal to or less than about 8% sucrose. In certain embodiments, the pharmaceutical formulation comprises equal to or less than about 8.5% sucrose. In certain embodiments, the pharmaceutical formulation comprises equal to or less than about 9% sucrose. In certain embodiments, the pharmaceutical formulation comprises equal to or less than about 9.5% sucrose. In certain embodiments, the pharmaceutical formulation comprises equal to or less than about 10% sucrose.

In certain embodiments, the pharmaceutical formulation comprises about 1-15% dextrose. In certain embodiments, the pharmaceutical formulation comprises about 1% dextrose. In certain embodiments, the pharmaceutical formulation comprises about 2% dextrose. In certain embodiments, the pharmaceutical formulation comprises about 3% dextrose. In certain embodiments, the pharmaceutical formulation comprises about 4% dextrose. In certain embodiments, the pharmaceutical formulation comprises about 5% dextrose. In certain embodiments, the pharmaceutical formulation comprises about 6% dextrose. In certain embodiments, the pharmaceutical formulation comprises about 7% dextrose. In certain embodiments, the pharmaceutical formulation comprises about 8% dextrose. In certain embodiments, the pharmaceutical formulation comprises about 9% dextrose. In certain embodiments, the pharmaceutical formulation comprises about 10% dextrose. In certain embodiments, the pharmaceutical formulation comprises about 11% dextrose. In certain embodiments, the pharmaceutical formulation comprises about 12% dextrose. In certain embodiments, the pharmaceutical formulation comprises about 13% dextrose. In certain embodiments, the pharmaceutical formulation comprises about 14% dextrose. In certain embodiments, the pharmaceutical formulation comprises about 15% dextrose.

In certain embodiments, the pharmaceutical formulation comprises equal to or greater than about 1% dextrose. In certain embodiments, the pharmaceutical formulation comprises equal to or greater than about 2% dextrose. In certain embodiments, the pharmaceutical formulation comprises equal to or greater than about 3% dextrose. In certain embodiments, the pharmaceutical formulation comprises equal to or greater than about 4% dextrose. In certain embodiments, the pharmaceutical formulation comprises equal to or greater than about 5% dextrose. In certain embodiments, the pharmaceutical formulation comprises equal to or greater than about 6% dextrose. In certain embodiments, the pharmaceutical formulation comprises equal to or greater than about 7% dextrose. In certain embodiments, the pharmaceutical formulation comprises equal to or greater than about 8% dextrose. In certain embodiments, the pharmaceutical formulation comprises equal to or greater than about 9% dextrose. In certain embodiments, the pharmaceutical formulation comprises equal to or greater than about 10% dextrose. In certain embodiments, the pharmaceutical formulation comprises equal to or greater than about 11% dextrose. In certain embodiments, the pharmaceutical formulation comprises equal to or greater than about 12% dextrose. In certain embodiments, the pharmaceutical formulation comprises equal to or greater than about 13% dextrose. In certain embodiments, the pharmaceutical formulation comprises equal to or greater than about 14% dextrose. In certain embodiments, the pharmaceutical formulation comprises equal to or greater than about 15% dextrose.

In certain embodiments, the pharmaceutical formulation comprises equal to or less than about 1% dextrose. In certain embodiments, the pharmaceutical formulation comprises equal to or less than about 2% dextrose. In certain embodiments, the pharmaceutical formulation comprises equal to or less than about 3% dextrose. In certain embodiments, the pharmaceutical formulation comprises equal to or less than about 4% dextrose. In certain embodiments, the pharmaceutical formulation comprises equal to or less than about 5% dextrose. In certain embodiments, the pharmaceutical formulation comprises equal to or less than about 6% dextrose. In certain embodiments, the pharmaceutical formulation comprises equal to or less than about 7% dextrose. In certain embodiments, the pharmaceutical formulation comprises equal to or less than about 8% dextrose. In certain embodiments, the pharmaceutical formulation comprises equal to or less than about 9% dextrose. In certain embodiments, the pharmaceutical formulation comprises equal to or less than about 10% dextrose. In certain embodiments, the pharmaceutical formulation comprises equal to or less than about 11% dextrose. In certain embodiments, the pharmaceutical formulation comprises equal to or less than about 12% dextrose. In certain embodiments, the pharmaceutical formulation comprises equal to or less than about 13% dextrose. In certain embodiments, the pharmaceutical formulation comprises equal to or less than about 14% dextrose. In certain embodiments, the pharmaceutical formulation comprises equal to or less than about 15% dextrose.

In certain embodiments, the pharmaceutical formulation comprises about 5 mg/mL Gamitrinib. In certain embodiments, the pharmaceutical formulation comprises about 2.5% DMSO. In certain embodiments, the pharmaceutical formulation comprises about 0.125% polysorbate 80. In certain embodiments, the pharmaceutical formulation comprises about 0.031% lecithin. In certain embodiments, the pharmaceutical formulation comprises about 1.25% sucrose. In certain embodiments, the pharmaceutical formulation comprises about 4.375% dextrose.

In certain embodiments, the pharmaceutical formulation is a solution of a suspension. In certain embodiments, the pharmaceutical formulation comprises a suspension of average particle size of about 50-500 nm. In certain embodiments, the pharmaceutical formulation comprises a suspension of average particle size of about 125, 130, 135, 140, 145, 150, 151, 152, 153, 154, 155, 156, 157, 158, 159, 160, 165, 170, and/or 175 nm. In certain embodiments, the pharmaceutical formulation comprises a suspension of average particle size equal to or greater than about 125, 130, 135, 140, 145, 150, 151, 152, 153, 154, 155, 156, 157, 158, 159, 160, 165, 170, and/or 175 nm. In certain embodiments, the pharmaceutical formulation comprises a suspension of average particle size equal to or less than about 125, 130, 135, 140, 145, 150, 151, 152, 153, 154, 155, 156, 157, 158, 159, 160, 165, 170, and/or 175 nm. In certain embodiments, the pharmaceutical formulation comprises a suspension of D(0.9) size of about 50-1000 nm. In certain embodiments, the pharmaceutical formulation comprises a suspension of D(0.9) size of about 200, 205, 210, 215, 220, 221, 222, 223, 224, 225, 226, 227, 228, 229, 230, 231, 232, 233, 234, 235, 240, 245, and/or 250 nm. In certain embodiments, the pharmaceutical formulation comprises a suspension of D(0.9) size equal to or greater than about 200, 205, 210, 215, 220, 221, 222, 223, 224, 225, 226, 227, 228, 229, 230, 231, 232, 233, 234, 235, 240, 245, and/or 250 nm. In certain embodiments, the pharmaceutical formulation comprises a suspension of D(0.9) size equal to or less than about 200, 205, 210, 215, 220, 221, 222, 223, 224, 225, 226, 227, 228, 229, 230, 231, 232, 233, 234, 235, 240, 245, and/or 250 nm.

In certain embodiments, the present disclosure provides a method of generating a Gamitrinib pharmaceutical formulation suitable for parenteral formulation, the method comprising subjecting to microfluidization a mixture of a DMSO-containing solution of Gamitrinib and a water-containing solution of polysorbate 80, lecithin (Lipoid S100), sucrose, and dextrose.

In certain embodiments, the method provides a solution of a suspension. In certain embodiments, the method provides a suspension of average particle size of about 50-500 nm. In certain embodiments, the method provides a suspension of average particle size of about 125, 130, 135, 140, 145, 150, 151, 152, 153, 154, 155, 156, 157, 158, 159, 160, 165, 170, and/or 175 nm. In certain embodiments, the suspension has an average particle size equal to or greater than about 125, 130, 135, 140, 145, 150, 151, 152, 153, 154, 155, 156, 157, 158, 159, 160, 165, 170, and/or 175 nm. In certain embodiments, the suspension has an average particle size equal to or less than about 125, 130, 135, 140, 145, 150, 151, 152, 153, 154, 155, 156, 157, 158, 159, 160, 165, 170, and/or 175 nm.

In certain embodiments, the method provides a suspension of D(0.9) size of about 50-1000 nm. In certain embodiments, the method provides a suspension of D(0.9) size of about 200, 205, 210, 215, 220, 221, 222, 223, 224, 225, 226, 227, 228, 229, 230, 231, 232, 233, 234, 235, 240, 245, and/or 250 nm. In certain embodiments, the suspension has D(0.9) size equal to or greater than about 200, 205, 210, 215, 220, 221, 222, 223, 224, 225, 226, 227, 228, 229, 230, 231, 232, 233, 234, 235, 240, 245, and/or 250 nm. In certain embodiments, the suspension has D(0.9) size equal to or less than about 200, 205, 210, 215, 220, 221, 222, 223, 224, 225, 226, 227, 228, 229, 230, 231, 232, 233, 234, 235, 240, 245, and/or 250 nm.

Method of Treating, Ameliorating and/or Preventing Cancer

In some aspects, the present invention is directed to a method of treating, ameliorating, and/or preventing cancer in a subject in need thereof.

In some embodiments, the method comprising administering to the subject Gamitrinib according to an administration protocol.

In some embodiments, the administration scheme includes a drug dosing phase. In some embodiments, the drug dosing phase last about one week, about two weeks, about three weeks, about four weeks, about one month, about six weeks, about two months, about three months, about four months, about five months, or about six months.

In some embodiments, the drug dosing phase includes a standard dosing phase. In some embodiments, during the standard dosing phase, the subject is administered with Gamitrinib at a dosage ranging from about 0.05 mg/kg per week to about 50 mg/kg per week, such as from about 0.1 mg/kg per week to about 25 mg/kg per week, about 0.2 mg/kg per week to about 10 mg/kg per week, about 0.3 mg/kg per week to about 8 mg/kg per week, about 0.5 mg/kg per week to about 5 mg/kg per week, about 0.6 mg/kg per week to about 4 mg/kg per week, about 0.7 mg/kg per week to about 3 mg/kg per week, about 0.8 mg/kg per week to about 2.5 mg/kg per week, about 1 mg/kg per week to about 2 mg/kg per week, about 0.05 mg/kg per week, about 0.1 mg/kg per week, about 0.2 mg/kg per week, about 0.4 mg/kg per week, about 0.5 mg/kg per week, about 0.75 mg/kg per week, about 1 mg/kg per week, about 1.5 mg/kg per week, about 2 mg/kg per week, about 3 mg/kg per week, about 5 mg/kg per week, about 7.5 mg/kg per week, about 10 mg/kg per week, about 15 mg/kg per week, about 20 mg/kg per week, about 30 mg/kg per week, or about 50 mg/kg per week.

In some embodiments, during the standard dosing phase, the subject is administered with Gamitrinib at a dosage that is equal to or greater than about 0.05 mg/kg per week, about 0.1 mg/kg per week, about 0.2 mg/kg per week, about 0.3 mg/kg per week, about 0.4 mg/kg per week, about 0.5 mg/kg per week, about 0.6 mg/kg per week, about 0.7 mg/kg per week, about 0.8 mg/kg per week, about 0.9 mg/kg per week, about 1 mg/kg per week, about 1.5 mg/kg per week, about 2 mg/kg per week, about 2.5 mg/kg per week, about 3 mg/kg per week, about 4 mg/kg per week, about 5 mg/kg per week, about 6 mg/kg per week, about 7 mg/kg per week, about 7.5 mg/kg per week, about 8 mg/kg per week, about 9 mg/kg per week, about 10 mg/kg per week, about 15 mg/kg per week, about 20 mg/kg per week, about 30 mg/kg per week, about 40 mg/kg per week, or about 50 mg/kg per week. In some embodiments, during the standard dosing phase, the subject is administered with Gamitrinib at a dosage that is equal to or less than about 50 mg/kg per week, about 40 mg/kg per week, about 30 mg/kg per week, about 25 mg/kg per week, about 20 mg/kg per week, about 15 mg/kg per week, about 10 mg/kg per week, about 8 mg/kg per week, about 7.5 mg/kg per week, about 6 mg/kg per week, about 5 mg/kg per week, about 4 mg/kg per week, about 3 mg/kg per week, about 2.5 mg/kg per week, about 2 mg/kg per week, about 1.5 mg/kg per week, about 1 mg/kg per week, about 0.9 mg/kg per week, about 0.8 mg/kg per week, about 0.75 mg/kg per week, about 0.7 mg/kg per week, about 0.6 mg/kg per week, about 0.5 mg/kg per week, about 0.4 mg/kg per week, about 0.3 mg/kg per week, about 0.2 mg/kg per week, about 0.1 mg/kg per week, or about 0.05 mg/kg per week.

In some embodiments, during the drug dosing phase, Gamitrinib is administered daily, every two days, three times per week, twice per week, once per week, or once every two weeks.

In some embodiments, the subject is administered with Gamitrinib for more than once in one week, and the dose of each administration is calculated by dividing the weekly dose by the times of administration in one week. In some embodiments, the subject is administered with Gamitrinib once every several weeks, and the dose of each administration is calculated by multiplying the weekly dose by the number of weeks for each administration.

In some embodiments, Gamitrinib is the only compound administered to the subject to treat, ameliorate and/or prevent cancer. As used herein, the term “compound administered to the subject to treat, ameliorate and/or prevent cancer” means a compound that is both able to treat, ameliorate and/or prevent cancer, and administered in dosages suitable for treating, ameliorating and/or preventing the cancer that Gamitrinib is being used to treat, ameliorate, and/or prevent.

In some embodiments, the drug dosing phase, in addition to the standard dosing phase, further includes an acceleration dosing phase prior to the standard phase. In some embodiments, the amount of Gamitrinib administered to the subject per week during the acceleration dosing phase is equal to or greater than the amount of Gamitrinib administered to the subject per week during the standard dosing phase. In some embodiments, the amount of Gamitrinib administered to the subject per week during the acceleration dosing phase is greater than the amount of Gamitrinib administered to the subject per week during the standard dosing phase.

In some embodiments, in addition to the drug dosing phase, the administration scheme further includes a recovery phase. In some embodiments, the recovery phase is after the dosing phase. In some embodiments, the subject is not administered Gamitrinib during the recovery phase. In some embodiments, during the recovery phase, the subject is allowed to recover from one, some, or all of the adverse effects caused by the administration of Gamitrinib during the dosing phase. In some embodiments, the recovery phase lasts about one week, about two weeks, about three weeks, about four weeks, about one month, about two months, about three months, about four months, about five months, or about six months.

In some embodiments, in addition to the drug dosing phase, the administration protocol further includes a predose phase. In some embodiments, the predose phase takes place before the drug dosing phase. In some embodiments, during the predose phase, baseline information of the subject is gathered such that the effectiveness of the Gamitrinib administration during the drug dosing phase can be evaluated, and/or adverse effects of the Gamitrinib can be observed/noted. In some embodiments, the baseline information includes weight, biomarker levels such as levels of cancer markers and/or inflammation markers, tumor size, location, grade and/or stage of the subject, and the like.

In some embodiments, the amount of Gamitrinib administered to the subject during the drug dosing phase is at or below the no observed adverse effect level (NOAEL) of Gamitrinib. In some embodiments, the amount of Gamitrinib administered to the subject during the drug dosing phase is lower than the highest non-severely toxic dose (HNSTD) of Gamitrinib. In some embodiments, the amount of Gamitrinib administered to the subject during the drug dosing phase is lower than the severely toxic dose. Referring to the “Example” section, the instant specification describes methods of determining the NOAEL, the HNSTD and the severely toxic dose, and the study described in “Example 1” section (“the first study”) determined the NOAELs of Gamitrinib in both Sprague-Dawley rats and beagle dogs, HNSTD in beagle dogs, as well as severely toxic dose in Sprague-Dawley rats. As such, one of ordinary skill in the art, reading in light of the instant specification, would be able to determine the NOAELs, HNSTDs and severely toxic doses of Gamitrinib in other mammalian subjects, without undue experimentation.

In some embodiments, the plasma concentration of Gamitrinib in the subject during the dosing phase ranges from about 0.1 ng/ml to about 10000 ng/ml, such as from about 1 ng/ml to about 5000 ng/ml, such as from about 10 ng/ml to about 2000 ng/ml, such as from 50 ng/ml to about 1000 ng/ml, such as from about 100 ng/ml to about 500 ng/ml. In some embodiments, the plasma concentration of Gamitrinib in the subject after 24 hours after one or more administration of Gamitrinib during the dosing phase ranges from about 0.1 ng/ml to about 10000 ng/ml, such as from about 1 ng/ml to about 5000 ng/ml, such as from about 10 ng/ml to about 2000 ng/ml, such as from 50 ng/ml to about 1000 ng/ml, such as from about 100 ng/ml to about 500 ng/ml.

In some embodiments, the plasma concentration of Gamitrinib in the subject after 24 hours after one or more administration of Gamitrinib during the dosing phase is equal to or greater than about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 120 ng/mL, about 140 ng/mL, about 160 ng/mL, about 180 ng/mL, about 200 ng/mL, about 220 ng/mL, about 240 ng/mL, about 260 ng/mL, about 280 ng/mL, about 300 ng/mL, about 320 ng/mL, about 340 ng/mL, about 360 ng/mL, about 380 ng/mL, about 400 ng/mL, about 420 ng/mL, about 440 ng/mL, about 460 ng/mL, about 480 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1200 ng/mL, about 1400 ng/mL, about 1600 ng/mL, about 1800 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, about 8000 ng/mL, about 8500 ng/mL, about 9000 ng/mL, about 9500 ng/mL, or about 10000 ng/mL.

In some embodiments, the plasma concentration of Gamitrinib in the subject after 24 hours after one or more administration of Gamitrinib during the dosing phase is equal to or less than about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 0.9 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 120 ng/mL, about 140 ng/mL, about 160 ng/mL, about 180 ng/mL, about 200 ng/mL, about 220 ng/mL, about 240 ng/mL, about 260 ng/mL, about 280 ng/mL, about 300 ng/mL, about 320 ng/mL, about 340 ng/mL, about 360 ng/mL, about 380 ng/mL, about 400 ng/mL, about 420 ng/mL, about 440 ng/mL, about 460 ng/mL, about 480 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1200 ng/mL, about 1400 ng/mL, about 1600 ng/mL, about 1800 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, about 8000 ng/mL, about 8500 ng/mL, about 9000 ng/mL, about 9500 ng/mL, or about 10000 ng/mL.

In some embodiments, the maximum serum concentration (Cₘₐₓ) of Gamitrinib in the subject during the dosing phase is about 2000 ng/mL or less, such as about 1800 ng/mL or less, about 1600 ng/mL or less, about 1500 ng/mL or less, about 1250 ng/mL or less, about 1000 ng/mL or less, about 900 ng/mL or less, about 750 ng/mL or less, about 600 ng/mL or less, about 500 ng/mL or less, about 400 ng/mL or less, about 300 ng/mL or less, about 200 ng/mL or less, or about 100 ng/mL or less.

In some embodiments, the maximum serum concentration (Cₘₐₓ) of Gamitrinib in the subject during the dosing phase is equal to about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 120 ng/mL, about 140 ng/mL, about 160 ng/mL, about 180 ng/mL, about 200 ng/mL, about 220 ng/mL, about 240 ng/mL, about 260 ng/mL, about 280 ng/mL, about 300 ng/mL, about 320 ng/mL, about 340 ng/mL, about 360 ng/mL, about 380 ng/mL, about 400 ng/mL, about 420 ng/mL, about 440 ng/mL, about 460 ng/mL, about 480 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1200 ng/mL, about 1400 ng/mL, about 1600 ng/mL, about 1800 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, about 8000 ng/mL, about 8500 ng/mL, about 9000 ng/mL, about 9500 ng/mL, or about 10000 ng/mL.

In some embodiments, the maximum serum concentration (Cₘₐₓ) of Gamitrinib in the subject during the dosing phase is equal to or greater than about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 120 ng/mL, about 140 ng/mL, about 160 ng/mL, about 180 ng/mL, about 200 ng/mL, about 220 ng/mL, about 240 ng/mL, about 260 ng/mL, about 280 ng/mL, about 300 ng/mL, about 320 ng/mL, about 340 ng/mL, about 360 ng/mL, about 380 ng/mL, about 400 ng/mL, about 420 ng/mL, about 440 ng/mL, about 460 ng/mL, about 480 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1200 ng/mL, about 1400 ng/mL, about 1600 ng/mL, about 1800 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, about 8000 ng/mL, about 8500 ng/mL, about 9000 ng/mL, about 9500 ng/mL, or about 10000 ng/mL.

In some embodiments, the maximum serum concentration (Cₘₐₓ) of Gamitrinib in the subject during the dosing phase is equal to or less than about 10 ng/mL, about 15 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 120 ng/mL, about 140 ng/mL, about 160 ng/mL, about 180 ng/mL, about 200 ng/mL, about 220 ng/mL, about 240 ng/mL, about 260 ng/mL, about 280 ng/mL, about 300 ng/mL, about 320 ng/mL, about 340 ng/mL, about 360 ng/mL, about 380 ng/mL, about 400 ng/mL, about 420 ng/mL, about 440 ng/mL, about 460 ng/mL, about 480 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, about 1000 ng/mL, about 1200 ng/mL, about 1400 ng/mL, about 1600 ng/mL, about 1800 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, about 8000 ng/mL, about 8500 ng/mL, about 9000 ng/mL, about 9500 ng/mL, or about 10000 ng/mL.

In some embodiments, the area under the curve during 24 hours (AUC₀₋₂₄) Gamitrinib in the subject after one or more administration of Gamitrinib during the dosing phase ranges from about 50 ng·hr/mL to about 5000 ng·hr/mL, such as from about 100 ng·hr/mL to about 2500 ng·hr/mL, from about 150 ng·hr/mL to about 1800 ng·hr/mL, from about 200 ng·hr/mL to about 1500 ng·hr/mL, from about 250 ng·hr/mL to about 2000 ng·hr/mL, from about 500 ng·hr/mL to about 1000 ng·hr/mL.

In some embodiments, the area under the curve during 24 hours (AUC₀₋₂₄) Gamitrinib in the subject after one or more administration of Gamitrinib during the dosing phase is equal to or greater than about 50 ng·hr/mL, about 100 ng·hr/mL, about 150 ng·hr/mL, about 200 ng·hr/mL, about 250 ng·hr/mL, about 500 ng·hr/mL, about 1000 ng·hr/mL, about 1500 ng·hr/mL, about 1800 ng·hr/mL, about 2000 ng·hr/mL, about 2500 ng·hr/mL, and/or about 5000 ng·hr/mL.

In some embodiments, the area under the curve during 24 hours (AUC₀₋₂₄) Gamitrinib in the subject after one or more administration of Gamitrinib during the dosing phase is equal to or less than about 50 ng·hr/mL, about 100 ng·hr/mL, about 150 ng·hr/mL, about 200 ng·hr/mL, about 250 ng·hr/mL, about 500 ng·hr/mL, about 1000 ng·hr/mL, about 1500 ng·hr/mL, about 1800 ng·hr/mL, about 2000 ng·hr/mL, about 2500 ng·hr/mL, and/or about 5000 ng·hr/mL.

In some embodiments, the subject is further administered with a compound for treating, ameliorating and/or preventing inflammation or renal complication(s). In some embodiments, the compound for treating, ameliorating and/or preventing the inflammation or renal complication(s) is administered before, during, and/or after the dosing phase. The first study described in “Example 1” section discovered that, when high dosages of Gamitrinib is administered to Sprague-Dawley rats or beagle dogs, inflammation and/or kidney tubular degeneration/regeneration were observed. As such, in some embodiments, compounds for treating, ameliorating and/or preventing inflammation or renal complication(s) are used to counter the adverse effects. Non-limiting examples of compounds for treating, ameliorating and/or preventing the inflammation are non-steroidal anti-inflammatory drugs (NSAIDs), corticosteroids, and the like. Non-limiting examples of NSAIDs include ibuprofen, meloxicam, naproxen, diclofenac, celecoxib, mefenamic acid, etoricoxib, indomethacin, aspirin, and the like. Non-limiting examples of corticosteroids include cortisone, prednisone, prednisolone, methylprednisolone, dexamethasone, betamethasone, hydrocortisone, and the like. Non-limiting examples of renal protective compounds include angiotensin-converting enzyme inhibitors (ACE inhibitors), angiotensin II receptor blockers (ARBs), and the like.

In some embodiments, Gamitrinib is formulated as a suspension. In some embodiments, Gamitrinib is formulated as a formulation suitable for parenteral administration. In some embodiments, Gamitrinib is formulated as a suspension suitable for parenteral administration.

In some embodiments, the cancer that is being treated, ameliorated and/or prevented by Gamitrinib and the administration scheme includes a carcinoma, a sarcoma, a lymphoma, a leukemia, a germ cell tumor, or combinations thereof. In some embodiments, the cancer includes a solid tumor, a lymphoma, or combinations thereof. In some embodiments, the solid tumor includes an advanced solid tumor. Examples of the cancer includes cancer or tumor of the lung, breast, epithelium, large bowel, rectum, testicle, gallbladder, bile duct, biliary tract, prostate, colon, stomach, esophagus, pancreas, liver, uterus, ovary, or brain. In some embodiments, the cancer include chronic myelogenoeous leukemia, B lymphoblastoid leukemia, breast adenocarcinoma, lung adenocarcinoma, prostate adenocarcinoma, gliobastoma, colon adenocarcinoma, and cervical carcinoma, haemangioma, Hodgkin's disease, large cell non-Hodgkin's lymphoma, malignant lymphoma, leukemia, polycythemia vera, neuroblastoma, retinoblastoma, myelodysplastic syndrome with refractory anemia, neuroblastoma, glioma, pheochromocytoma, soft tissue sarcoma, maxillary cancer, lingual cancer, lip cancer, mouth cancer, melanoma, or non-melanoma skin cancer. In some embodiments, the cancer includes a cervical cancer, a breast cancer, a prostate cancer, a lung cancer, an epithelial carcinoma, a colorectal cancer, a Burkitt lymphoma, a myeloid leukemia, a leukemic monocyte lymphoma, or the like.

In some embodiments, the subject is a mammal, such as a human.

Kit for Treating, Ameliorating and/or Preventing Cancer

In some aspects, the present invention is directed to a kit for treating, ameliorating and/or preventing cancer.

In some embodiments, the kit includes Gamitrinib and an instruction manual. In some embodiments, the instruction manual instructs a method of treating, ameliorating and/or preventing cancer according to any method contemplated elsewhere herein.

In some embodiments, the kit further includes a compound for treating, ameliorating and/or preventing inflammation and/or renal complication(s). In some embodiments, the instruction manual further instructs to administer the compound for treating, ameliorating and/or preventing inflammation or the renal protective compound, such as according to the method as described elsewhere herein.

### Combination Therapies

The compounds useful within the methods described herein can be used in combination with one or more additional therapeutic agents useful for treating, ameliorating, and/or preventing cancer. These additional therapeutic agents may comprise compounds that are commercially available or synthetically accessible to those skilled in the art. These additional therapeutic agents are known to treat or reduce the symptoms of a cancer.

In some embodiments, the Gamitrinib used herein is combined with one or more additional anticancer agents. In some embodiments, the one or more additional anticancer agents include a MAPK inhibitor. Anticancer agents, such as MAPK inhibitors suitable for combining with Gamitrinib to treat, ameliorate and/or prevent cancer is described in, for example, US Patent Application No. 2018/0228740 A1. The entirety of this reference is hereby incorporated herein by reference.

In certain embodiments, the compounds described herein can be used in combination with radiation therapy. In other embodiments, the combination of administration of the compounds described herein and application of radiation therapy is more effective in treating or preventing cancer than application of radiation therapy by itself. In yet other embodiments, the combination of administration of the compounds described herein and application of radiation therapy allows for use of lower amount of radiation therapy in treating the subject.

In various embodiments, a synergistic effect is observed when a compound as described herein is administered with one or more additional therapeutic agents or compounds. A synergistic effect may be calculated, for example, using suitable methods such as, for example, the Sigmoid-Eurax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The administration schemes, such as dosages, as well as formulations of Gamitrinib useful for the method of treating, ameliorating and/or preventing cancer is detailed elsewhere herein. Any description elsewhere herein, however; should not be construed to be limiting. One of ordinary skill in the art, reading in light of the instant specification, would understand that additional administration/dosage/formulations are useful for the methods and kits of the instant invention, as well.

The regimen of administration may affect what constitutes an effective amount. The therapeutic formulations may be administered to the subject either prior to or after the onset of a cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a cancer in the patient. An effective amount of the therapeutic compound necessary to achieve a therapeutic effect may vary according to factors such as the state of the disease or disorder in the patient; the age, sex, and weight of the patient; and the ability of the therapeutic compound to treat a cancer in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of cancer in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time-controlled release of a compound as described herein by pH-independent swelling.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The dosages of Gamitrinib useful for treating, ameliorating and/or preventing cancer are detailed elsewhere herein. The descriptions elsewhere herein should not be construed as limiting. One of ordinary skill in the art, reading in light of the instant specification, would understand that additional dosages of Gamitrinib and/or other compounds described herein are also useful for the methods and kits herein.

The therapeutically effective amount or dose of a compound described herein depends on the age, sex and weight of the patient, the current medical condition of the patient and the progression of a cancer in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Preclinical Characterization of Gamitrinib

Mitochondria are key tumor drivers, but their suitability as a therapeutic target is unknown. The study described in this section (“the first study”) reports the preclinical characterization of Gamitrinib (GA mitochondrial matrix inhibitor), a first-in-class anticancer agent that couples the Heat Shock Protein-90 (Hsp90) inhibitor 17-allylamino-geldanamycin (17-AAG) to the mitochondrial-targeting moiety, triphenylphosphonium. Formulated as a stable (≥24 weeks at −20° C.) injectable suspension produced by microfluidization (<200 nm particle size), Gamitrinib (>99.5% purity) is heavily bound to plasma proteins (>99%), has intrinsic clearance from liver microsomes of 3.30 mL/min/g and minimally penetrates a Caco2 intestinal monolayer. Compared to 17-AAG, Gamitrinib has slower clearance (85.6±5.8 mL/min/kg), longer t½ (12.2±1.55 h), mean AUC₀₋ₜ of 783.1±71.3 h·ng/mL, and unique metabolism without generation of 17-AG. Concentrations of Gamitrinib that trigger tumor cell killing (IC₅₀˜1-4 μM) do not affect cytochrome P450 isoforms CYP1A2, CYP2A6, CYP2B6, CYP2C8 or ion channel conductance (Nav1.5, Kv4.3/KChIP2, Cav1.2, Kv1.5, KCNQ1/mink, HCN4, Kir2). Twice weekly IV administration of Gamitrinib to Sprague-Dawley rats or beagle dogs for up to 36 d is feasible. At dose levels up to 5 (rats)- and 12 (dogs)-fold higher than therapeutically effective doses in mice (10 mg/kg), Gamitrinib treatment is unremarkable in dogs with no alterations of clinical-chemistry parameters, heart function or tissue histology and causes occasional inflammation at the infusion site and mild elevation of serum urea nitrogen in rats (>10 mg/kg/dose). Therefore, targeting mitochondria for cancer therapy is feasible and well tolerated. A publicly funded, first-in-human phase I clinical trial of Gamitrinib in patients with advanced cancer is ongoing (ClinicalTrials.gov NCT04827810, see “Example 2” section).

### Example 1-1: Related Information

The rewiring of metabolic pathways, including in mitochondria is a ubiquitous cancer trait important for disease progression. Accordingly, exploitation of mitochondrial bioenergetics, buffering of reactive oxygen species (ROS) and inhibition of cell death pathways have been implicated in tumor growth, acquisition of metastatic competence, and resistance to conventional and molecular therapy. On this basis, mitochondria may provide a unique therapeutic target in cancer, suitable to disable key pathways of tumor maintenance, regardless of genetic makeup or driver mutation(s). As a result, multiple mitochondrial-targeted anticancer agents, or mitocans, have been developed.

In addition, there is evidence that mitochondria may be uniquely ‘wired’ in cancer compared to normal tissues, potentially enabling a broader therapeutic window. One example is the ubiquitous dependence, or ‘addiction’ of tumor mitochondria to a heightened protein folding environment, essential to buffer the proteotoxic stress invariably associated with tumor growth, in vivo. Mechanistically, this is accomplished with the selective accumulation of molecular chaperones, including Heat Shock Protein-90 (Hsp90) and its homolog TNF Receptor-Associated Protein 1 (TRAP1), as well as AAA+ proteases in tumor mitochondria, compared to normal tissues. In turn, Hsp90 chaperoning stabilizes a multifunctional mitochondrial proteome in cancer, including key metabolic regulators, lowers ROS and prevents cell death.

Although targeting chaperone-directed proteostasis shows promising antitumor activity, pharmacologically, this pathway escapes inhibition by small molecule Hsp90 antagonists with geldanamycin (GA) or non-GA backbones as these agents fail to accumulate in mitochondria. To overcome this conundrum, the first study generated Gamitrinib (GA mitochondrial matrix inhibitor), a first-in-class, mitochondrial-targeted inhibitor of organelle protein folding that links the GA Hsp90 inhibitor 17-allylamino-geldanamyicn (17-AAG, Tanespymicin) to an efficient mitochondrial-import carrier, triphenylphosphonium (TPP). Owing to its unique chemical structure, Gamitrinib selectively accumulates in mitochondria with a 106-fold enrichment compared to cytosol by mass spectrometry of isolated subcellular fractions. Once in mitochondria, Gamitrinib triggers acute proteotoxic stress, shuts off multiple organelle functions, including bioenergetics, and delivers potent anticancer activity with IC₅₀ of 0.16-29 μM in an NCI 60 cell line screen, including cell lines representative of common malignancies, such as colon adenocarcinoma (IC₅₀, 0.35-29 μM), breast adenocarcinoma (IC₅₀, 0.16-3.3 μM) and melanoma (IC₅₀, 0.36-2.7 μM). Strong anticancer activity was also seen in combination regimens of Gamitrinib plus molecular therapy in models of epithelial and hematopoietic malignancies.

Here, the first study reports the preclinical characterization of Gamitrinib as the first, subcellularly-directed antagonist of mitochondrial proteostasis. Based on these findings, a publicly funded, first-in-human phase I clinical trial of Gamitrinib in patients with advanced cancer is currently ongoing (ClinicalTrials.gov, NCT04827810).

### Example 1-2: Materials and Methods

### Cell Lines

Caco-2 and HEK293 cells were obtained from the American Type Culture Collection (ATCC, Manasass, Va.) and grown in culture according to the supplier's specifications. In some experiments, HEK293 cells were stably transfected with hERG cDNA and polyclonal cultures were maintained in the presence of 250 μg/ml geneticin (G418). Cells were maintained in medium containing 10% fetal calf serum at 37° C. in 5% CO2 and plated on 35 mm dishes at least 24 h prior to the experiment.

Chemical synthesis and analysis of Gamitrinib

The complete chemical synthesis, HPLC profile, and mass spectrometry of Gamitrinib (GA mitochondrial matrix inhibitor) is described in Kang et al. (J Clin Invest 2009; 119:454-64). The structure of Gamitrinib is combinatorial and contains the Hsp90 inhibitor 17-AAG linked to triphenylphosphonium as a mitochondrial-targeting carrier. The bulk Gamitrinib powder is stored at −20° C. in the dark.

### Formulation Development of Gamitrinib

A sequential three-step process was utilized to prepare Good Laboratory Practice (GLP) working solutions of Gamitrinib (5 mg/mL) for preclinical studies. The formulation workflow is as follows: Step 1—Solubilization of Gamitrinib powder in DMSO (2.5%); Step 2—Dilution in 1.25% (w/v) Polysorbate 80, 0.31% (w/v) Lecithin (Lipoid 5100) and 12.5% (w/v) Sucrose (10%) in sterile water for injection; Step 3—Dilution in 5% dextrose (87.5%). Therefore, the final Gamitrinib formulation is ˜5 mg/mL Gamitrinib, 2.5% DMSO, 0.125% Polysorbate 80, 0.031% Lecithin, 1.25% Sucrose and 4.375% Dextrose. For Good Manufacturing Practice (GMP) studies, a Gamitrinib Injectable Suspension (GIS) was prepared by microfluidization. Gamitrinib stock solutions prepared as above were passed through a microfluidizer (Dyhydromatics, Acton, Mass.) with rate of flow set at low, medium, and high. At the end of GIS processing, microfluidization was continued at reduced pressure (2000 psi) for 1-2 min. The parameters for GIS microfluidization as are follows: ratio of organic to aqueous phase (DMSO: aqueous vehicle 1:40 v/v); filter membrane materials (PTFE for DMSO, cellulose acetate for aqueous vehicle); microfluidizer pressure during mixing organic and aqueous phase (28,000 psi); post-mixing pressure in microfluidizer (2000 psi); temperature inside interaction chamber (0 to −10° C. before initiating microfluidization). The final Gamitrinib Injectable Suspension (GIS) after microfluidization is 4.86 mg/mL, with average particle size of 154 nm, D(0.9) size of 229 nm, pH 6.0.

### Gamitrinib Pharmacokinetics (PK) in Rat Plasma

Sprague-Dawley rats (n=3) were administered a single intravenous (IV) dose of 1 mL Gamitrinib (5 mg/kg) formulated as described above without added microfluidization. Blood samples were collected via lateral tail vein using K2EDTA as an anticoagulant at 0.083, 0.25, 0.5, 1, 2, 4 and 24 h post-dose, chilled on ice and centrifuged at 5000 rpm for 10 min. Aliquots (0.2 mL) of each plasma sample were stored frozen at −20° C. For analysis, 50 μL aliquots of study samples were mixed with 50 μL of ACN/H₂O (50:50), extracted by protein precipitation using ACN containing the internal standard Gamitrinib-d₁₅ (100 ng/mL), and aliquots of supernatant were mixed with deionized water for Gamitrinib determination. Batch samples under analysis included a calibration curve, a matrix blank (blank rat plasma), a reagent blank, a control zero (blank rat plasma spiked with internal standard), and duplicate QC samples at 3 concentration levels (low, medium, and high) in addition to the study samples. Within each batch, the study samples were bracketed by calibration standards or QC samples. The lowest calibration standard served to evaluate system suitability at the beginning of each batch. In all batches, the system suitability samples displayed adequate separation and acceptable peak shapes, retention times, and signal-to-noise ratios. Overall, the concentration of Gamitrinib was measured in 21 rat plasma samples in 1 analytical batch using LC-MS/MS data acquisition on a Shimadzu Nexera LC system coupled with an AB Sciex Triple Quad 5500 mass spectrometer. Chromatograms were integrated using Analyst 1.6.2 software. A weighted (1/x², x=concentration) linear regression was used to generate the calibration curve for Gamitrinib. The concentration of Gamitrinib was calculated using the peak area ratio of analyte to internal standard based on the standard curve. The mean, standard deviation, precision, accuracy, and assay variability were calculated using Microsoft Excel.

### Toxicity in Rats

Ninety-six male and female Sprague-Dawley rats (6-7 weeks old) were received from Charles River Laboratories, (Raleigh, N.C.) and catheterized via a femoral vein prior to arrival. Animals were acclimated in individual stainless-steel cages with access to water and Certified Rodent Diet #2014C (Envigo RMS) ad libitum for 6 d prior to study initiation. Environmental controls were set to maintain a temperature range of 20 to 26° C., a relative humidity range of 30 to 70%, eight or greater air changes/h, and a 12-h light/dark cycle. Animals were infused with sterile isotonic (0.9%) saline for a minimum of 4 d (0.20 mL/h for males or 0.15 mL/h for females) during the predose phase. At initiation of dosing, animals were 8 to 9 weeks old, and body weights ranged from 283 to 341 g for males and 188 to 238 g for females. Gamitrinib formulated as indicated above was administered by IV infusion on d 4, 8, 11, 15, 18, 22, 25, and 29 of the dosing phase at 1, 10, 25 mg/kg/dose and dose volume of 5 mL/kg. The vehicle control administered with the same schedule contained 2.5% DMSO, 0.125% polysorbate 80, 0.031% lecithin, 1.25% sucrose and 4.375% dextrose. Animals were checked twice daily throughout the duration of the study for mortality, abnormalities, and signs of pain or distress. Detailed observations were conducted for each animal up to two times during the predose phase and for each toxicity animal prior to dosing on d 1, 8, 15, 22, and 29 of the dosing phase and on d 1, 7, and 14 of the recovery phase. Blood samples were collected on d. 1, 4 and 29 of the dosing phase 5 min, 1 h and 24 h post-dose, maintained on chilled cryoracks and centrifuged within 1 h of collection. Tissue samples harvested from each animal were embedded in paraffin, sectioned, and slides were prepared and stained with hematoxylin and eosin.

### Toxicity in Beagle Dogs

Eighteen male and female (5-6 mo old) purebred beagle dogs (Cumberland, Va.) were acclimated for 47 d (20 to 26° C., relative humidity 30-70%, 10 or greater air changes/h, and a 12-h light/dark cycle) prior to study initiation. Animals were given water ad libitum and Certified Canine Diet #5007 (PMI Nutrition International Certified LabDiet®) for 4 to 5 h each day. At study initiation, animal body weights ranged from 8.6 to 11.5 kg for males and 6.0 to 9.4 kg for females. At least 1 week prior to initiation of dosing, animals were fasted overnight, anesthetized, and a catheter attached to a subcutaneous vascular access port was surgically implanted into a jugular vein. Animals were acclimated to infusion jackets prior to catheter implantation surgery. Animals were infused with sterile saline at a dose rate of 5 mL/h when the vascular access ports were accessed and when animals were connected to the infusion system (except when dosed with the test or vehicle control article). The patency of each catheter was checked as needed. Prednisolone tablets were administered orally at 2 mg/kg the night prior to dosing. Diphenhydramine (5 mg/kg; IM injection 0.1 mL/kg, 50 mg/mL) was administered prior to dosing and post the start of infusion (d 1 only). Male and female dogs received dose levels of Gamitrinib of 1.25, 3.33 and 6.25 mg/kg/dose formulated as indicated above during a 1 h infusion on d 1, 8, 11, 15, 18, 22, 25, 29, 32, and 36 at a volume of 2 mL/kg. The vehicle control was as above. During dosing on d 1, multiple animals, including controls, were noted with clinical observations of swollen head/body, hypoactivity, twitching, red skin, and/or vocalization due to vehicle components. Dosing was stopped, and 23 of 24 animals were administered additional diphenhydramine, 11 were administered Flunixin meglumine and buprenorphine, and 6 were administered acepromazine. Symptoms subsided in all animals after completion of these interventions. As a result of these observations, the polysorbate levels, which were increased from 0.025% (w/v) to 0.125% (w/v) to minimize precipitation, were returned to the original concentrations; 2 mg/kg prednisone was administered orally the night prior to dosing; and the diphenhydramine pretreatment dose was updated to 5 mg/kg 15 to 30 min prior to dosing. Detailed observations were conducted for each animal up to seven times during the predose phase, prior to dosing on d 1, 8, 15, 22, 29, and 36 of the dosing phase, and on d 1, 7, and 14 of the recovery phase. Blood samples (1 mL) were collected via the cephalic vein 5, 15, and 30 min and 1, 4, 8, and 24 h post the end of infusion on d 8 and 36 of the dosing phase. Formalin-fixed and paraffin-embedded tissue samples collected at the end of the study (d 39) were processed for histologic examination.

### Statistical Analysis

Levene's test was used to test for equality of variances between groups. Where Levene's test was not significant (P>0.05), ANOVA was conducted; where Levene's test was significant (1210.05), a rank transformation was applied before the ANOVA was conducted. Where the group effect from the ANOVA was significant (P≤10.05), comparisons between each treated group and the control were made using Dunnett's t-test. If the ANOVA was not significant (P>0.05), no further analyses were conducted.

### Chemicals and Reagents

Quercetin, ketoconazole, phenacetin, bupropion, paclitaxel, diclofenac, midazolam, testosterone, tolbutamide, β-nicotinamide adenine dinucleotide phosphate sodium salt hydrate (NADP), D-glucose 6-phosphate sodium salt (G6P), glucose-6-phosphate dehydrogenase (G6PDH), digoxin, sucrose, dextrose, propranolol, lucifer yellow (LY), warfarin, HEPES, D-glucose, terfenadine, polysorbate 80 and dimethyl sulfoxide (DMSO) were obtained from Sigma-Aldrich (St. Louis, Mo.). Fluvoxamine, tranylcypromine, clopidogrel, and sulfaphenazole were obtained from Cayman Chemicals (Ann Arbor, Mich.). Lecithin was purchased from Lipoid (Lipoid S100). 17-amino-geldanamycin (17-AG) was obtained from Toronto Research Chemicals (Toronto, Canada). Potassium phosphate buffer (KPi) was prepared at 100 mM, pH 7.4. NADPH regenerating system, cofactor (5X) was prepared with NADP (1.7 mg/mL, 2.22 mM), G6P (7.8 mg/mL, 27.6 mM) and G6DPH (2.0 U/mL) in 100 mM KPi. Human plasma and pooled human liver microsomes were obtained from Bioreclamation IVT (Baltimore, Md.). HBSS, acetonitrile, BupH phosphate buffered saline packs and rapid equilibrium dialysis (RED) devices were obtained from Thermo Fisher Scientific (Waltham, Mass.). Stop solution was prepared with ACN containing 100 ng/mL tolbutamide.

### Plasma Protein Binding

Gamitrinib or control stock solutions (5 mM) were prepared in DMSO, and further diluted in DMSO/water (1:1, v/v) to 0.2 mg/mL. Spiked plasma samples were prepared by adding 12 μL of each working solution into 1.2 mL of blank plasma to a final concentration of 2 μg/mL. Plasma protein binding incubations were performed in triplicate by adding 500 μL of PBS into each white chamber and 300 μL of spiked plasma into each red chamber of the RED device. The device was covered with a sealing tape and placed on an orbital shaker at a shaking speed of 100 rpm at 37° C. in 5% CO2. After a 4-h incubation, a 40 μL plasma sample was transferred from each red chamber to the wells of a 96-well plate containing 160 μL of PBS. A 160 μL PBS aliquot from each white chamber was transferred to the wells of the same plate containing 40 μL of blank plasma. After matching the matrix for all samples, 400 μL aliquots of stop solution were added to each well of the 96-well sample plate. The plate was vortexed for 3 min at 1700 rpm followed by centrifugation at 3500 rpm for 15 min. A 100 μL aliquot of supernatant from each well was transferred to a new 96-well sample plate and further mixed with 100 μL of Milli-Q (ultrapure) water before analysis by liquid chromatography—tandem mass spectrometry (LC-MS/MS).

### CYP Inhibition in Liver Microsomes

Working solutions of Gamitrinib at concentrations of 0, 0.033, 0.1, 0.33, 1, 3.3, 10 and 33 μM were incubated with human liver microsomes (0.1 mg/mL) with an NADPH regenerating system. Positive controls were added to human liver microsomes in parallel reactions with serial concentrations of 0, 0.033, 0.1, 0.33, 1, 3.3, 10, 33 μM (clopidogrel, CYP2B6) or 0.0033, 0.01, 0.033, 0.1, 0.33, 1, 3.3 μM (fluvoxamine, CYP1A2; tranylcypromine, CYP2A6; quercetin, CYP2C8; sulfaphenazole, CYP2C9; ketoconazole, CYP3A4). After addition of prototypical CYP substrates in 250 μL, samples were incubated for 10 min (CYP1A2, CYP2B6, CYP2C9, and CYP3A4) or 20 min (CYP2C8) at 37° C. in 5% CO₂. After addition of 250 μL stop solution, the plate was vortexed for 3 min at 1700 rpm and centrifuged at 3500 rpm for 15 min. Samples were analyzed for metabolite formation by LC-MS/MS. The results were expressed using the peak area ratio of analyte to internal standard, based on the calibration curves. The inhibitor concentration that resulted in 50% inhibition (IC₅₀) of enzyme activity was calculated by fitting the % control activity vs. concentration to the following equation:

$$y = \frac{Range}{[ 1 + {( \frac{x}{IC 50} )}^{S} ]}$$

where Range=fitted uninhibited value, s=slope factor, x=inhibitor concentration, y=% control activity.

### Ion Channel Screening

To test the effect of Gamitrinib (10 μM) on channel conductance, human Nav1.5 currents were evoked by stepping from a holding potential of −120 mV to −20 mV for 150 msec (50 msec inter-pulse interval) for a total of 26 pulses. The parameters measured were the maximum inward current evoked on stepping to −20 mV from the 1^{st} and 26^{th} pulse. Human Kv4.3/KChIP2 currents were evoked from a holding potential of −80 mV by a series of four 500 msec pulses to 0 mV using a 1000 msec interval between pulses. The parameter measured was the amplitude of the outward current 50 msec after the onset of the 4^{th} voltage step to 0 mV. Human Cav1.2 currents were evoked by 2 pulses to −10 mV from a holding potential of −100 mV. The parameters measured were the maximum inward currents evoked on stepping to −10 mV from the holding potential of −100 mV for the 1^{st} and 2^{nd} pulse. Human Kv1.5 currents were evoked by a single pulse from a holding potential of −80 mV to a potential of 0 mV for 4 sec before returning to −80 mV. The parameters measured were the maximum amplitude of outward currents evoked at the beginning of the voltage pulse and at the end of the voltage step from −80 mV to 0 mV. Human KCNQ1/minK currents were evoked by a single pulse delivered from a holding potential of −80 mV to +60 mV for 4 sec before returning to −80 mV. The parameter measured was the maximum outward current evoked on stepping to +60 mV from the holding potential of −80 mV. hERG currents were evoked by a three-pulse protocol where voltage was first stepped to +40 mV for two sec from a holding potential of −80 mV to inactivate hERG channels. The voltage is then stepped back to −50 mV for two sec to evoke a tail current prior to returning to the holding potential for 1 sec. The parameter measured was the amplitude of the 3^{rd} pulse tail current upon stepping back to −50 mV after the step to +40 mV. Human HCN4 currents were evoked by a single pulse from a holding potential of −30 mV to a potential of −110 mV for 4 sec prior to returning to −30 mV. The parameter measured was the maximum inward current evoked upon stepping to −110 mV from the holding potential of −30 mV. Human Kir2.1 currents were evoked from a holding potential of −20 mV by a series of 10 500 msec pulses to −120 mV using a 200 msec interval between pulses. The parameters measured were the amplitudes of the instantaneous inward currents evoked on stepping to −120 mV for the 1^{st} pulse and the maximum inward current at the end of the 10^{th} hyperpolarizing pulse. For all experiments, data were filtered for seal quality, seal drop, and current amplitude.

### Patch Clamp Recording

The extracellular solution for whole cell patch clamp recordings was 137 mM NaCl, 1.2 mM MgCl₂, 5.4 mM KCl, 10 mM glucose, 10 mM HEPES and 2 mM CaCl₂ (305 mOsm), pH 7.4. The intracellular solution was 140 mM KCl, 2.1 mM MgCl, 5 mM EGTA, 10 mM HEPES and 5 mM Na₂ATP (295 mOsm), pH 7.2. For electrophysiology recording, a micropipette was pulled from borosilicate glass with the pipette tip resistance between 3˜5 MΩ. For each experiment, polyclonal HEK293 cells stably transfected with hERG cDNA and maintained in 250 μg/ml Geneticin (G418) were plated on 35 mm dishes on the microscope stage and a commercial patch clamp amplifier was used for the whole cell recordings. The tail currents were evoked at 22° C. once every 30 sec by a 3 sec-50 mV repolarizing pulse following a 2 sec+50 mV depolarizing pulse with a hold voltage of −80 mV. A 50 msec depolarized pulse to −50 mV at the beginning of the voltage protocol served as a baseline for calculating the amplitude of the peak tail current. The hERG currents were allowed to stabilize over a 3 min period in the presence of vehicle alone prior to Gamitrinib application. The cells were kept in the test solution until the peak tail current was stable (<5% change) for ˜5 sweeps or for a maximum of 6 min, whichever came first. Peak tail amplitudes were plotted as a function of the sweep number. Five peak tail current measurements at the steady state before Gamitrinib application were averaged and used as the control current amplitude. Four or five peak tail current measurements at the steady state after Gamitrinib application were averaged and used as the remaining current amplitude after inhibition by the test article. The % inhibition of the test article was calculated from the following equation:

% inhibition=1−(remaining current amplitude)/(control current amplitude)*100.

Electrocardiography Studies

Electrocardiograms (ECGs) were obtained from unanesthetized beagle dogs administered Gamitrinib at dose levels of 1.25 mg/kg/dose (3 males and 3 females), 3.33 mg/kg/dose (3 males and 3 females) and 6.25 mg/kg/dose (5 males and 5 females) once during the predose phase, 1 to 2 h post end of infusion on d 32 of the dosing phase, and on d 11 of the recovery phase. At each time point, eight-lead ECGs were continuously recorded for at least 30 sec. At least five consecutive ECG waveforms and the associated RR intervals deemed most representative were selected for quantitative analysis at each time point. The ECG interval measurements (PR, QRS, QT, QTc, RR, and heart rate) were made on a single lead. The measured waveforms at each time point were averaged and reported. The QTc interval was calculated using the Fridericia (QT/[RR]^{1/3}) method. The RR interval was used to derive the heart rate and in QT interval correction. Qualitative assessments included normal sinus rhythm variations, abnormal sinus rhythms, conductance or repolarization abnormalities, bradycardia, and tachycardia. Group comparisons of ECG data (PR, QRS, QT, QTc, and heart rate) were analyzed using ANOVA.

### Bidirectional Caco2 Cell Monolayer Permeability

Caco2 cells (2×10⁴/well) were seeded onto apical chambers of a 24-multiwell insert plate and incubated at 37° C. in 5% CO₂. After 21 d, the transepithelial electrical resistance (TEER) in ohms was read for each insert well as a quality control for monolayer integrity (>900 ohms or 300 ohms cm²). The cell monolayers were incubated with 400 μL transport buffer on the apical (A) and 1.2 mL on the basolateral (B) sides for 30 min. After preincubation, dosing solution was added to the apical (400 μL, A-to-B, donor) or basolateral sides (1.2 mL, B-to-A, donor) or receiving buffer to the apical (400 μL, B-to-A, receiver) or basolateral sides (1.2 mL, A-to-B, receiver) for 90 min at 37° C. in 5% CO₂. At the end of the incubation, 300 μL from A and B sides were transferred to a receiving plate, centrifuged for 15 min at 3500 rpm and analyzed by LC-MS/MS. The integrity of each monolayer was assessed with Lucifer Yellow (LY) at Em 430 nm and Ex 540 nm (batch results, 0.41; acceptance criteria, <1% of dosing solution) and TEER values (batch results, 443; acceptance criteria, >300). Pₐₚₚ·A>B (nm/s) was calculated with Equation 1.

$$Papp = \frac{Vr \cdot Cr}{A \cdot t \cdot Cdo} \qquad Equation 1$$

where Cr is concentration in receiver well, Cd0 is initial dosing concentration, Vr is the receiver well volume; A is the membrane surface area (0.3 cm²) and T is the incubation time (120 min*60s/min).
Papp Efflux Ratio (ER) was calculated with Equation 2.

$$ER = \frac{Papp , B > A}{Papp , A > B} \qquad Equation 2$$

Propranolol (high permeability marker, non-P-gp substate) and digoxin (P-gp substrate) were used as controls.

### Example 1-3: Results

### Chemical Synthesis and Formulation Development of Gamitrinib

The chemical synthesis of Gamitrinib containing the Hsp90 inhibitor 17-AAG linked to the mitochondrial-targeting carrier, triphenylphosphonium via an hexylamine linker is described in Kang et al. (J Clin Invest 2009; 119:454-64). Clinical-grade (GMP compliant) Gamitrinib synthesized as described in FIG. 5A has the chemical formula C₅₂H₆₅F₆N₃O₈P₂ (>99.5% purity by UPLC), is purple solid (TM.795) and crystalline by X-ray powder diffraction, with a molecular weight of 1036.03. 500 MHz 1H NMR spectrum (DMSO-d6), 125 MHz ¹³C NMR spectrum (DMSO-d6) and 282 MHz ¹⁹F NMR spectrum (DMSO-d6) are all consistent with structure. The water content is 0.7% (Karl Fischer analysis) and the residual solvent concentrations (methanol, DCM, MTBE and DIPEA) are all below limit of quantification (BLOQ, <3000, <600, <5000 and <3000 ppm, respectively).

Gamitrinib is formulated using a three-step dilution process described in the Materials and Methods section. A sterile, Gamitrinib Injectable Suspension (GIS) is prepared for clinical use using microfluidization with the schematic flowchart shown in FIG. 5B. The resulting GIS has average particle size of 154 nm and D(0.9) size of 229 nm, pH 6.0. When stored frozen at −20° C., the GIS shows no significant changes in stability or particle size distribution upon analysis at 1, 2, 4, 8, 12 and 24 weeks after manufacturing (Table 1-1).

TABLE 1-1
Stability and particle size distribution of Gamitrinib Injectable Suspension (GIS).

| | Time of storage (2 ml GIS in 4 ml sterile glass vials at −20° C. | | | | | | |
|---|---|---|---|---|---|---|---|
| | 0 | 1 wk | 2 wk | 4 wk | 8 wk | 12 wk | 24 wk |
| Appearance | Purple | Purple | Purple | Purple | Purple | Purple | Purple |
| | susp | susp | susp | susp | susp | susp | susp |
| Particle size (nm) | 200 | 198 | 204 | 203 | 205 | 203 | 212 |
| F/T particle size (nm) | | 197 | 209 | 207 | 216 | 208 | 225 |
| Assay (mg/ml) | 4.41 | 4.80 | 4.57 | 4.43 | 4.47 | 4.47 | 4.61 |
| Recovery (% over T0) | 100 | 109 | 103 | 100 | 101 | 101 | 104 |

Susp, suspension;
F/T, freeze-thaw;
wk, week.

### In Vitro Toxicity

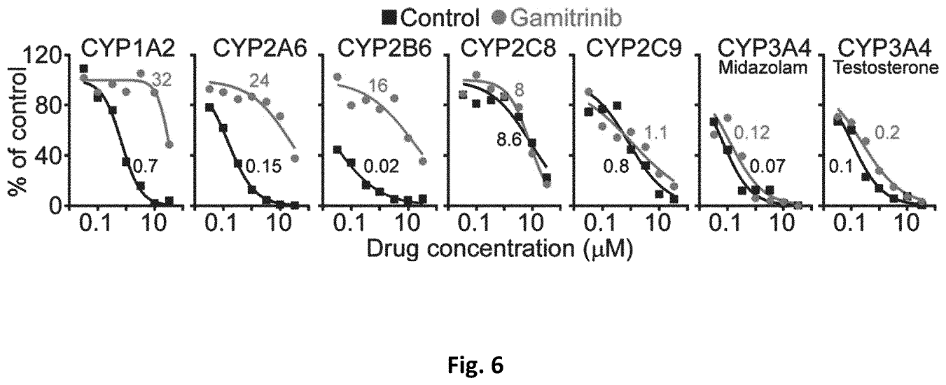

Concentrations of Gamitrinib that trigger tumor cell killing in culture (IC₅₀−1-4 μM) did not inhibit cytochrome P450 isoforms CYP1A2 (IC₅₀, 32.9 μM), CYP2A6 (IC₅₀, 24 μM), CYP2B6 (IC₅₀, 16 μM), and CYP2C8 (IC₅₀, 8 μM) (FIG. 6). Conversely, Gamitrinib inhibited CYP2C9 (IC₅₀, 1.1 μM) and CYP3A4 (IC₅₀, 0.12-0.2 μM) (FIG. 6).

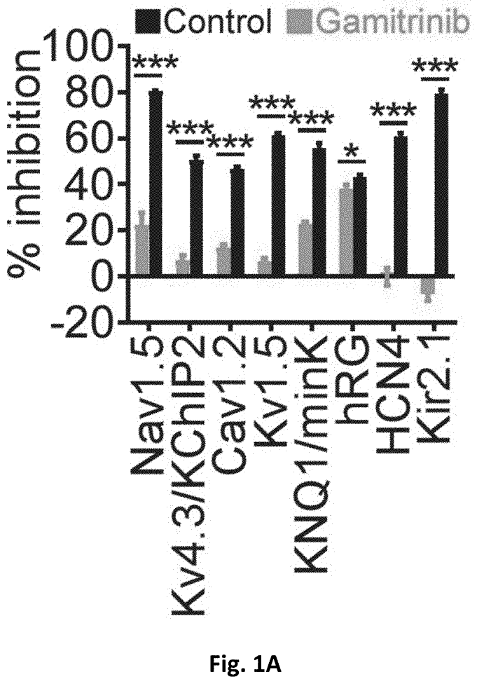

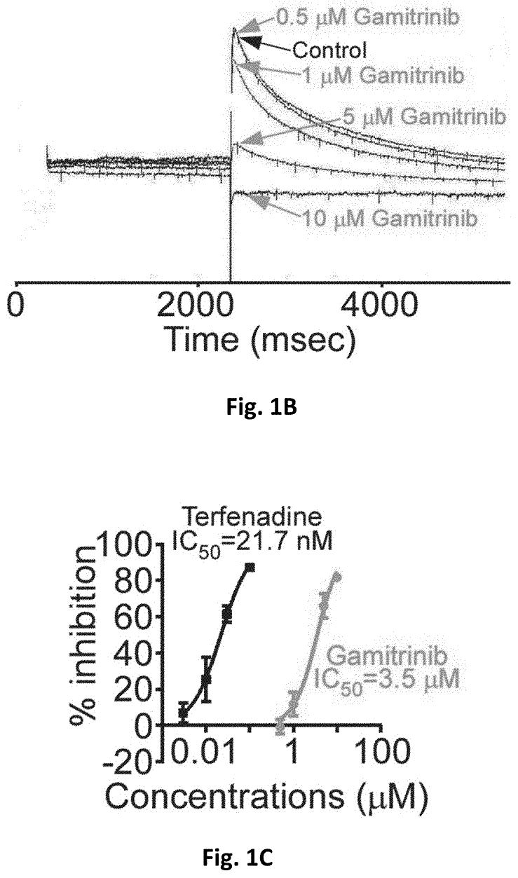

When analyzed for ion channel conductance, high concentrations of Gamitrinib (10 μM) inhibited Nav1.5 currents by 22.3±5.3% (control, 80.3±0.5%; n=3, pulse 26), Kv4.3/KChIP2 by 6.8±2.2% (control 50.5±2; n=13), Cav1.2 by 12.2±1.5% (control, 46.8±0.7%; n=34, pulse 2), Kv1.5 by 6.6±1.3% (control 61±1%; n=15), KCNQ1/mink by 22.5±1.1% (control 55.7±2.4%; n=16), hERG by 37.9±1.7% (control 42.9±1.2%; n=16), HCN4 by −0.2±3.7% (control 60.8±1.2%; n=16) and Kir2.1 by −7.3±3.4% (control 79.2±1.9%; n=15, pulse 10) (FIG. 1A). A potential effect of Gamitrinib on hERG currents was further studied in patch-clamp experiments in transfected HEK293 cells (FIG. 1B). Here, concentrations of Gamitrinib of 0.5, 1, 5 and 10 μM inhibited hERG currents by −0.68±3.95%, 11.71±6.51%, 65.95±6.78%, and 81.83±1.88%, respectively (mean±SD) (FIG. 1B), resulting in a Gamitrinib IC₅₀ of hERG inhibition of 3.5 μM (terfenadine IC₅₀ 21.7 nM) (FIG. 1C).

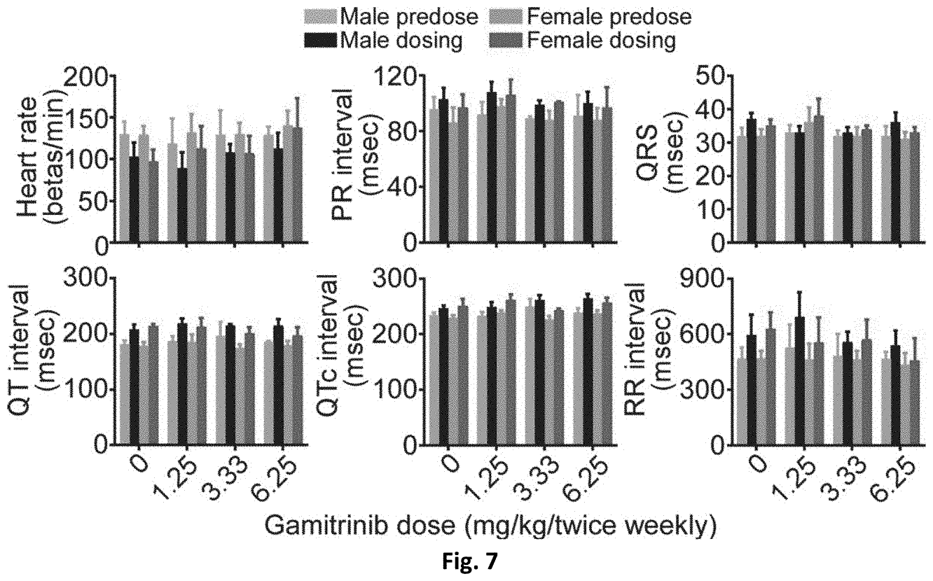

To further characterize a potential cardiac toxicity of Gamitrinib, electrocardiography studies were carried out in beagle dogs administered IV Gamitrinib at dose levels of 1.25, 3.3, and 6.25 mg/kg/dose twice weekly for 36 d plus a 14-d recovery period (FIG. 7). In this analysis, one out of 5 male dogs administered Gamitrinib at 6.25 mg/kg/dose exhibited a small (7%) prolongation of QTc interval (17 msec) on d 32 of the dosing phase, which reversed during the recovery phase. No Gamitrinib-related prolongation of QTc interval was observed in female dogs administered 6.25 mg/kg/dose or in both sexes administered 1.25 or 3.33 mg/kg/dose (FIG. 7). No Gamitrinib-related ECG changes in PR interval, QRS duration, QT interval, or heart rate were observed on d 32 of the dosing phase in animals administered 1.25, 3.33 or 6.25 mg/kg/dose or on d 11 of the recovery phase in animals administered 6.25 mg/kg/dose. No other rhythm abnormalities or qualitative ECG changes were observed (FIG. 7).

### PK Studies

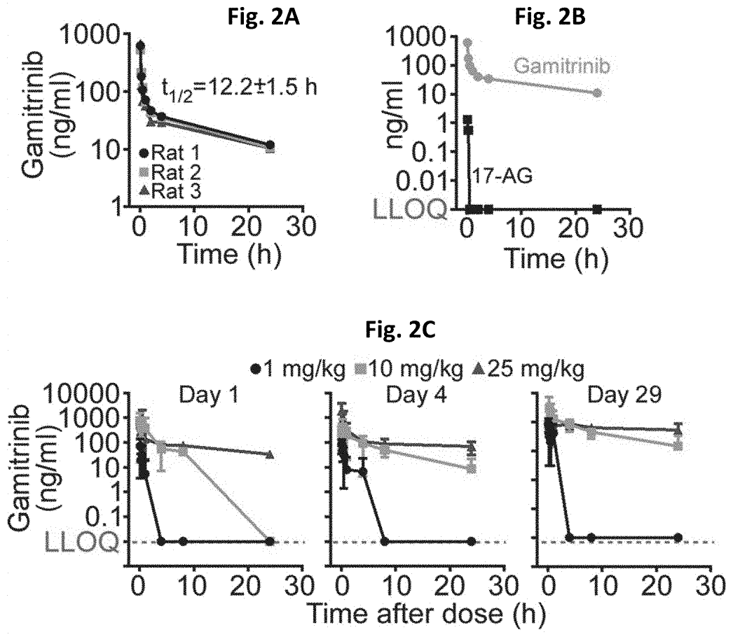

After IV administration (5 mg/kg) to Sprague-Dawley rats (n=3), the mean Gamitrinib Cₘₐₓ was 1175.807 ng/mL (FIG. 2A), with mean volume of distribution at steady state (Vₛₛ) of 65.471 L/kg, medium to slow clearance at 85.656±5.856 ml/min/kg and mean terminal phase half-life (t_{1/2}) of 12.25±1.55 h (Table 1-2). Mean AUC₀₋ₜ and AUC_{INF} values were 783.199 and 976.002 hr·ng/mL, respectively (Table 1-2). Gamitrinib metabolism in rats did not generate detectable levels of 17-(amino)-17-demethoxygeldanamicyin (17-AG) (FIG. 2B and Table 1-3), a key metabolite of 17-AAG processing, in vivo. IV administration of Gamitrinib to Sprague-Dawley rats at dose levels of 1, 10 or 25 mg/kg/dose twice weekly for 29 d resulted in increased Cₘₐₓ values from 1 to 25 mg/kg/dose followed by bi-exponential decline (FIG. 2C). CL_{SS} values ranged from 84.83 to 131.33 mL/min/kg and Vₛₛ values from 12.2 to 90.0 L/kg ford 4 and d 29. Gamitrinib Cₘₐₓ and AUC₀₋₂₄ values were similar on d 4 and d 29, indicating no drug accumulation after multiple doses. Accumulation ratio values ranged from 0.045 to 1.04 for Cₘₐₓ and from 0.344 to 1.33 for AUC₀₋₂₄.

TABLE 1-2
Gamitrinib (5 mg/kg IV) PK in Sprague-Dawley rats.

| PK | | | | | | | CV |
|---|---|---|---|---|---|---|---|
| Parameters | Unit | Rat 1 | Rat 2 | Rat 3 | Mean | SD | (%) |
| t_{1/2} | h | 11.623 | 11.107 | 14.022 | 12.250 | 1.555 | 12.7 |
| Cₘₐₓ | ng/ml | 1153.712 | 829.668 | 1544.041 | 1175.807 | 357.699 | 30.4 |
| CL | mL/min/kg | 79.233 | 87.035 | 90.700 | 85.656 | 5.856 | 6.8 |
| MRT | h | 5.539 | 5.411 | 5.524 | 5.492 | 0.0702 | 1.3 |
| Vz | L/Kg | 79.715 | 83.678 | 110.085 | 91.159 | 16.509 | 18.1 |
| Vss | L/Kg | 58.230 | 60.052 | 78.131 | 65.471 | 11.001 | 16.8 |
| AUCₗₐₛₜ | h · ng/mL | 851.441 | 789.045 | 709.112 | 783.199 | 71.344 | 9.1 |
| AUC_{INF} | h · ng/mL | 1051.752 | 957.472 | 918.784 | 976.002 | 68.394 | 7.0 |

AUC_{INF}; area under concentration vs. time curve from time 0 to infinity;
AUCₗₐₛₜ, area under concentration vs. time curve from time 0 to last quantifiable concentration;
Cₘₐₓ, maximum observed concentration;
CL, clearance;
MRT, mean residence time;
t_{1/2}, terminal half-life;
Vz, apparent volume of distribution;
Vss, volume of distribution at steady-state

TABLE 1-3
Time course of Gamitrinib and 17-AG plasma concentrations after
IV administration of Gamitrinib (5 mg/kg) to Sprague-Dawley rats

| Time | Animal | Collection | Dose | Gamitrinib | 17-AG |
|---|---|---|---|---|---|
| (min) | No. | Day | (mg/kg) | (ng/ml) | (ng/mL) |
| 5 | 1 | 1 | 5 | 622.199 | BLOQ |
| 15 | 1 | 1 | 5 | 180.951 | 0.561 |
| 30 | 1 | 1 | 5 | 106.381 | BLOQ |
| 60 | 1 | 1 | 5 | 71.445 | BLOQ |
| 120 | 1 | 1 | 5 | 46.657 | BLOQ |
| 240 | 1 | 1 | 5 | 36.816 | BLOQ |
| 1440 | 1 | 2 | 5 | 11.946 | BLOQ |
| 5 | 2 | 1 | 5 | 526.229 | 1.519 |
| 15 | 2 | 1 | 5 | 211.686 | BLOQ |
| 30 | 2 | 1 | 5 | 114.230 | BLOQ |
| 60 | 2 | 1 | 5 | 65.903 | BLOQ |
| 120 | 2 | 1 | 5 | 43.125 | BLOQ |
| 240 | 2 | 1 | 5 | 34.776 | BLOQ |
| 1440 | 2 | 2 | 5 | 10.511 | BLOQ |
| 5 | 3 | 1 | 5 | 674.432 | 1.113 |
| 15 | 3 | 1 | 5 | 128.663 | BLOQ |
| 30 | 3 | 1 | 5 | 67.204 | BLOQ |
| 60 | 3 | 1 | 5 | 55.991 | BLOQ |
| 120 | 3 | 1 | 5 | 29.914 | BLOQ |
| 240 | 3 | 1 | 5 | 28.905 | BLOQ |
| 1440 | 3 | 2 | 5 | 10.365 | BLOQ |

BLOQ, below limit of quantification.

### Plasma Protein Binding, Stability, Microsome Clearance and Intestinal Penetration

Gamitrinib was heavily bound to plasma proteins (99.3±0.07%) with an average free fraction of 0.7±0.07%, comparable to control warfarin (bound, 98.3±0.22%, free fraction, 1.68±0.22%). The stability of Gamitrinib in human plasma was 91.4% with an average recovery of 82.8±3% (warfarin, 88.3±2.9%). At a concentration of 0.5 μM, the elimination rate constant (k) of Gamitrinib in phase I, cytochrome P450-mediated human liver microsome metabolism was 0.041 (control Midazolam, k=0.04) with half-life (t½) of 16.7 min (Midazolam, t½=17 min) and intrinsic clearance (CLint) of 3.30 mL/min/g (Midazolam, 3.23 mL/min/g). Gamitrinib showed negligible penetration across a monolayer of Caco2 intestinal cells with an apparent permeability coefficient (P_{app′}) of 1.90 nm/s in the A-to-B direction and 10.94 nm/s in the B-to-A direction with a Pₐₚₚ Efflux Ratio (ER) of 5.77 (Table 1 μL).

TABLE 1-4
Gamitrinib permeability in a Caco2 intestinal cell monolayer assay

| | Pₐₚₚ, | Pₐₚₚ, | Pₐₚₚ Efflux | P_{exact}, | P_{exact}, | P_{exact} |
|---|---|---|---|---|---|---|
| Test | A > B | B > A | Ratio | A > B | B > A | Efflux |
| Compound | (nm/s) | (nm/s) | (ER) | (nm/s) | (nm/s) | Ratio |
| Gamitrinib | 1.90 | 10.94 | 5.77 | 1.97 | 10.92 | 5.54 |
| Propranolol | 186.12 | 162.49 | 0.87 | 209.35 | 170.12 | 0.81 |
| Digoxin | 5.47 | 114.80 | 21.00 | 4.13 | 87.64 | 21.21 |

### Toxicity in Sprague-Dawley Rats

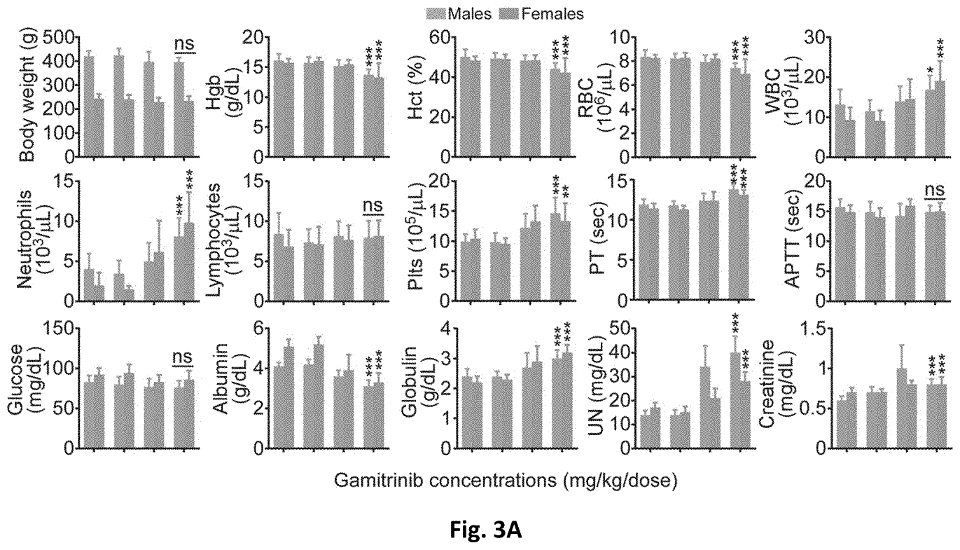

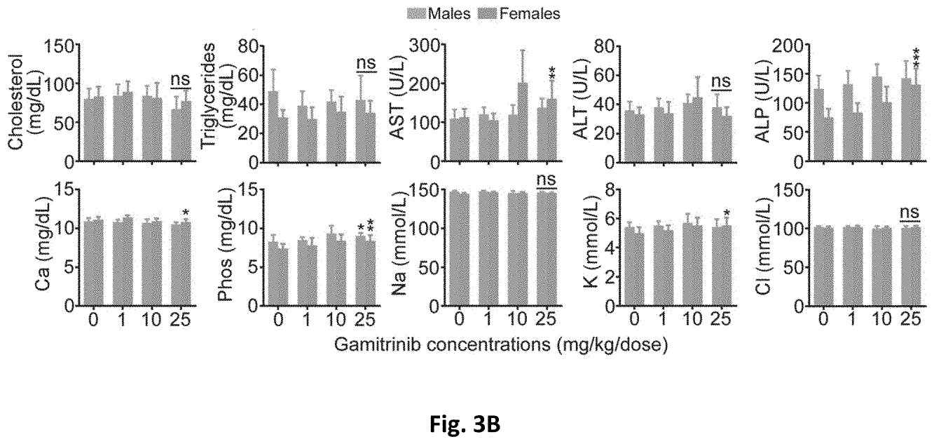

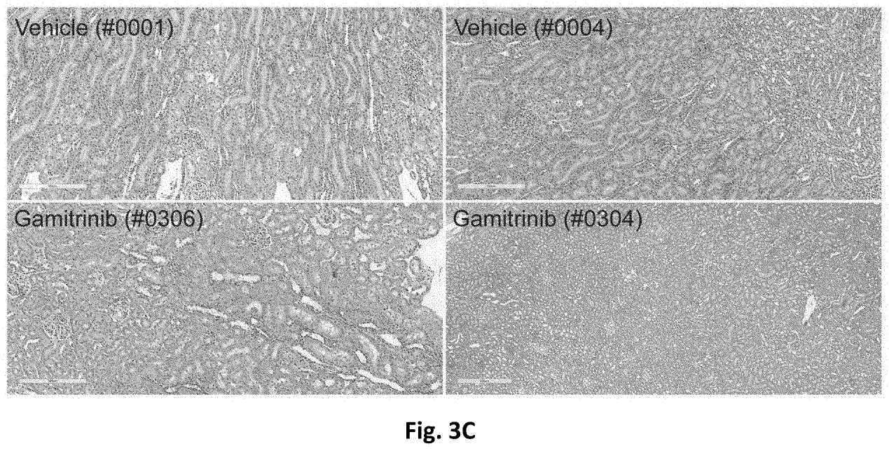

Male rats administered Gamitrinib IV (1 h infusion) at dose levels of 1, 10 or 25 mg/kg/dose twice weekly on d 1, 4, 8, 11, 15, 18, 22, 25, and 29 (dosing phase) exhibited a small, fully recoverable and not adverse reduction in mean body weights at 10 (−5.5%) or 25 (−5.7%) mg/kg/dose (FIGS. 3A-3C). Gamitrinib-related clinical observations involved animals administered 10 mg/kg/dose, and included inguinal swelling, piloerection, hypoactivity, and sensitivity to touch at the infusion site. This correlated with microscopic findings of mixed cell inflammation at the catheter/infusion site, which increased in incidence and/or severity in animals administered 10 mg/kg/dose (both sexes) and persisted through recovery. Alterations in clinical chemistry parameters, such as mildly to moderately higher neutrophil and platelet (Plts) counts, minimally prolonged partial thromboplastin time (PT), lower albumin, higher globulin, and alkaline phosphatase concentrations were observed at the highest Gamitrinib dose level tested (FIGS. 3A-3B) and likely related to inflammation, accompanied by histologic evidence of spleen and liver extramedullary hematopoiesis. Minimally to mildly higher serum urea nitrogen (UN) and creatinine concentrations in animals receiving Gamitrinib at 25 mg/kg/dose (FIGS. 3A-3C) correlated with increased kidney weight and microscopic findings of tubular degeneration/regeneration (FIG. 3C), which persisted to the end of the recovery phase. No effects on urinalysis or ophthalmic changes were identified. Gamitrinib-related mortality due to severe inflammation and marked hemorrhage at the infusion site occurred in two males and one female administered 25 mg/kg/dose and one male administered 10 mg/kg/dose. All other toxicity animals survived to their scheduled sacrifice. Gamitrinib-related mortality also occurred in two toxicokinetic females administered 10 mg/kg/dose and one toxicokinetic female administered 25 mg/kg/dose. Based on these findings, the no observed adverse effect level (NOAEL) of Gamitrinib in rats is 1 mg/kg/dose, corresponding to Cₘₐₓ and AUC₀₋₂₄ values of 87.1 ng/mL and 174 ng·hr/mL, respectively, on d 29 of dosing. Due to non-severely toxic effects or mortality in fewer than 10% of the animals administered 10 mg/kg/dose, the severely toxic dose in 10% of the animals (STD 10) is 10 mg/kg/dose, corresponding to Cₘₐₓ and AUC₀₋₂₄ values of 311 ng/mL and 1300 ng·hr/mL, respectively, on d 29.

### Toxicity in Beagle Dogs

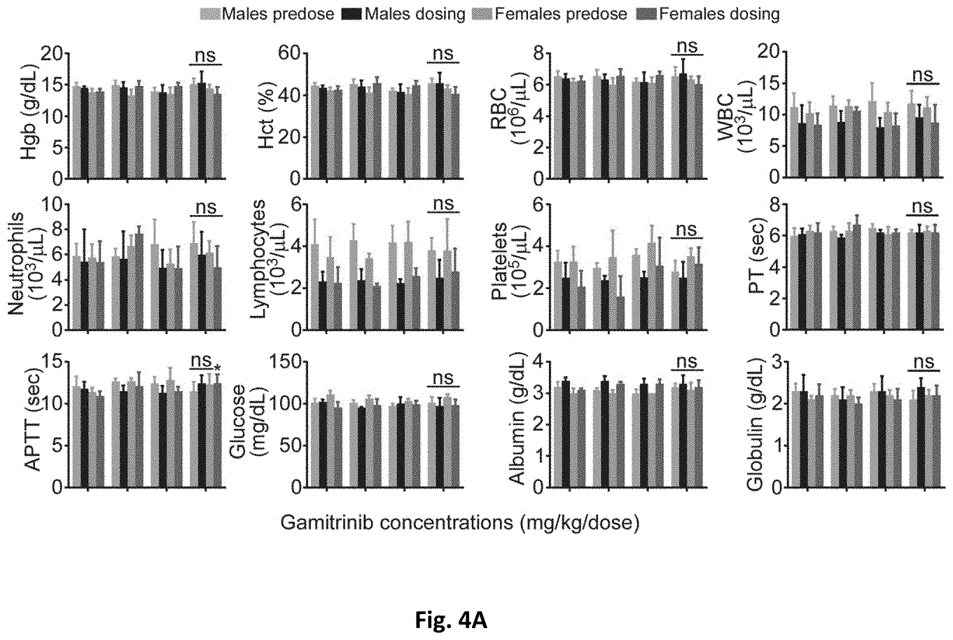

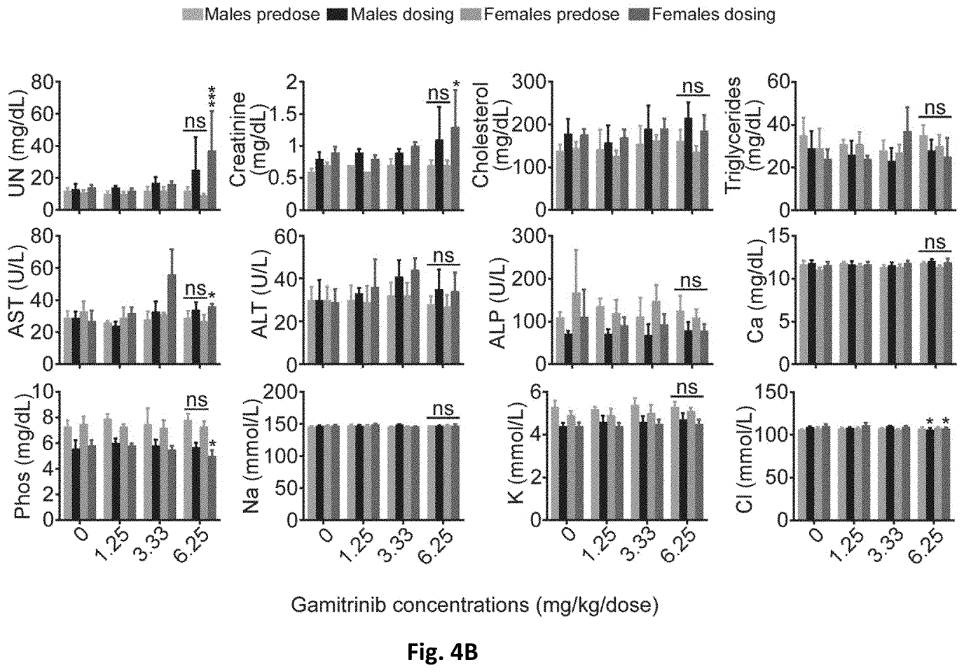

Male and female beagle dogs administered IV Gamitrinib at dose levels of 1.25, 3.33 and 6.25 mg/kg/dose on d 1, 8, 15, 22, 29, and 36 of the dosing phase showed no alterations in body weight or other clinical observations (Table 1-5). Bone marrow and liver parameters were unremarkable in all group levels, and only a trend of increased serum urea nitrogen and creatinine was observed in animals (both sexes) receiving the highest dose level of Gamitrinib of 6.25 mg/kg/dose (FIGS. 4A-4B). This correlated with microscopic finding of slight to moderate kidney tubular degeneration/regeneration, which was reversible during the recovery period. In addition, similar findings were present in one recovery sacrifice control male making their relationship to Gamitrinib uncertain. No Gamitrinib-related changes in organ weights were observed and electrolyte, calcium and phosphorus levels were unchanged in the various groups (FIGS. 4A-4B). Catheter and infusion site findings were similar in control and Gamitrinib-treated animals. Based on these findings, the NOAEL of Gamitrinib in dogs was 3.33 mg/kg/dose (Cₘₐₓ, 560±404 ng/mL; AUC₀₋₂₄, 1740±713 ng·h/mL on d 36 of dosing, both sexes). In the absence of effects on the overt well-being of the animals and evidence of reversibility of Gamitrinib-related findings, 6.25 mg/kg/dose is considered the highest non-severely toxic dose (HNSTD). This dose level corresponds to Cₘₐₓ and AUC₀₋₂₄ values of 1260±556 ng/mL and 3290±1090 ng·h/mL, respectively (both sexes), on d 36 of the dosing phase.

TABLE 1-5
Body weight alterations (Kg) in beagle dogs after Gamitrinib
IV infusion (dosing phase). N = number of animals.

| Days | 0 mg/kg | 1.25 mg/kg | 3.33 mg/kg | 6.25 mg/kg |
|---|---|---|---|---|
| | MALES Gamitrinib doses (IV infusion) | | | |
| 1 | 10.1 ± 0.83 | 10.3 ± 0.91 | 10.1 ± 1.1 | 9.9 ± 0.83 |
| | (N = 5) | (N = 3) | (N = 3) | (N = 5) |
| 8 | 10 ± 0.75 | 10.2 ± 1.04 | 10 ± 1.04 | 9.8 ± 0.85 |
| | (N = 5) | (N = 3) | (N = 3) | (N = 5) |
| 15 | 9.9 ± 0.66 | 10.1 ± 0.93 | 9.9 ± 1.15 | 9.5 ± 0.57 |
| | (N = 5) | (N = 3) | (N = 3) | (N = 5) |
| 22 | 10.1 ± 0.73 | 10.3 ± 0.69 | 10.2 ± 1.21 | 9.2 ± 0.57 |
| | (N = 5) | (N = 3) | (N = 3) | (N = 5) |
| 29 | 10 ± 0.66 | 10.3 ± 0.75 | 10.1 ± 1.21 | 9.2 ± 0.46 |
| | (N = 5) | (N = 3) | (N = 3) | (N = 5) |
| 36 | 10.1 ± 0.76 | 10.5 ± 0.52 | 10 ± 1.36 | 9.1 ± 0.58 |
| | (N = 5) | (N = 3) | (N = 3) | (N = 5) |
| | FEMALES Gamitrinib doses (IV infusion) | | | |
| 1 | 7.5 ± 0.92 | 7.9 ± 1.34 | 7.6 ± 0.9 | 7.7 ± 0.57 |
| | (N = 5) | (N = 3) | (N = 3) | (N = 5) |
| 8 | 7.4 ± 0.89 | 7.8 ± 1.49 | 7.6 ± 0.9 | 7.7 ± 0.62 |
| | (N = 5) | (N = 3) | (N = 3) | (N = 5) |
| 15 | 7.3 ± 0.93 | 7.8 ± 1.64 | 7.6 ± 1.06 | 7.4 ± 0.54 |
| | (N = 5) | (N = 3) | (N = 3) | (N = 5) |
| 22 | 7.3 ± 1.03 | 8 ± 1.53 | 7.5 ± 0.75 | 7.2 ± 0.6 |
| | (N = 5) | (N = 3) | (N = 3) | (N = 5) |
| 29 | 7.5 ± 0.94 | 8.2 ± 1.42 | 7.6 ± 0.7 | 7.3 ± 0.58 |
| | (N = 5) | (N = 3) | (N = 3) | (N = 5) |
| 36 | 7.6 ± 0.97 | 8.4 ± 1.55 | 7.5 ± 0.8 | 7.3 ± 0.59 |
| | (N = 5) | (N = 3) | (N = 3) | (N = 5) |

### Example 1-4: Discussion

In the first study, it has been shown that Gamitrinib, a first-in-class mitocan inhibitor of mitochondrial protein folding can be synthesized as clinical-grade material, formulated as a sterile, stable injectable suspension, and administered IV to two animal species for up to 36 d. In line with its unique mechanism of action of subcellular organelle targeting, Gamitrinib drug-like properties differ from those of non-mitochondrial-targeted 17-AAG (Tanespimycin) with slower clearance, longer half-life, and unique metabolism without generation of 17-AG. Finally, prolonged IV administration of Gamitrinib is essentially unremarkable in beagle dogs and causes occasional inflammation at the infusion site and modest alterations of kidney function in Sprague-Dawley rats.

When administered to dogs, concentrations of Gamitrinib up to 12.8-fold higher than therapeutically active doses in mice (10 mg/kg) did not elicit the extensive alterations of bone marrow, liver, and gastrointestinal tract functions seen with non-mitochondrial targeted 17-AAG and its derivatives, 17-DMAG and IPI-504. As the only reportable observation in the studies, the highest dose level of Gamitrinib of 6.25 mg/kg caused only modest elevation of urea nitrogen and creatinine concentrations with microscopic evidence of kidney tubule degeneration/regeneration, which was reversed during a 14-d recovery phase. A potential cardiac liability of Gamitrinib, prompted by the role of Hsp90 in hERG protein folding was also unremarkable. Gamitrinib IC₅₀ for hERG conductance in patch-clamp studies (3.5 μM) was up to 20-fold higher than the IC₅₀ of inhibition of tumor growth observed in an NCI 60-cell line screen, and only 1 out of 10 animals exhibited a small, 7% prolongation of QTc interval (17 msec) fully reversed during the recovery phase. In the rat study, Gamitrinib concentrations (25 mg/kg) up to 5-fold higher than the efficacious antitumor dose in mice, produced only occasional inflammation at the infusion/catheter site, accompanied by mild elevation of urea nitrogen and evidence of kidney tubule degeneration/regeneration.

In addition to improved safety, Gamitrinib exhibited unique drug-like properties compared to 17-AAG. These included slower clearance (85.65±5.85 mL/min/kg), much longer terminal phase half-life (t_{1/2}, 12.25±1.55 h), and no generation of 17-AG, a key metabolite of 17-AAG processing. The structural basis for the increased Gamitrinib stability in vivo is currently not known. However, these data are reminiscent of the greater stability of 17-DMAG compared to 17-AAG, and it is possible that the presence of the triphenylphosphonium side chain reduces the oxidative metabolism of Gamitrinib, causing slower drug clearance and longer t_{1/2}.

Based on the findings of the first study, the feasibility and safety of Gamitrinib is evaluated in a first-in-human, phase I clinical trial in patients with advanced cancer as part of a publicly funded academic effort (NCT04827810).

### Example 2: Phase I Clinical Trial of Gamitrinib

Based on the results of the first study of Example 1 and other data, a clinical trial of Gamitrinib is performed. This clinical trial is also described in ClinicalTrials.gov, with the identifier NCT04827810. The entirety of this reference is hereby incorporated herein by reference.

### Example 2-1: Study Description

The study described herein (“the second study”) is a first-in-human, phase I, open-label, non-randomized dose-escalation and dose-expansion study with the primary objective to determine the safety profile of small molecule, mitochondrial-targeted Hsp90 inhibitor, Gamitrinib, including identification of dose-limiting toxicities (DLT) and maximum tolerated dose (MTD) in patients with advanced cancers. A secondary objective of the second study is to determine the recommended dose and regimen(s) for a phase II study. This study is based on preclinical data demonstrating the anticancer activity, unique mechanism of action and preliminary safety of Gamitrinib.

In the dose-finding portion of the second study, Gamitrinib formulated in Lipoid S100®-based formulation is administered as a 1-hour IV infusion once weekly for four weeks as 28-day treatment cycles. Up to 36 patients is enrolled in the dose-escalation component of the study based on anticipated cohorts. The starting dose is 10 mg, corresponding to allometric scaling) from the most sensitive species (rats) in the 29-day GLP toxicology and toxicokinetic studies with 14-day recovery period of Gamitrinib. Dose-escalation follows a 3+3 design. Six patients are enrolled in the dose-expansion component of the study at MTD for the purpose of exploring pharmacodynamic effects via tumor pre and on-therapy biopsies.

TABLE 2-1
Study Description

| Condition or disease | Intervention/treatment | Phase |
|---|---|---|
| Lymphoma | Drug: Gamitrinib | Phase 1 |
| Advanced Solid Tumor | | |

### Example 2-2: Study Design

The study design of the second study is described in Table 2-2 below:

TABLE 2-2
Study design

| | |
|---|---|
| Study Type | Interventional (Clinical Trial) |
| Enrollment | 42 participants |
| Allocation | Non-Randomized |
| Intervention Model | Sequential Assignment |
| Masking | None (Open Label) |
| Primary Purpose | Treatment |

### Example 2-3: Arms and Interventions

The arms and interventions of the second study is described below in Table 2-3.

| Arm | Intervention/treatment |
|---|---|
| Experimental: Dose −1 | Drug: Gamitrinib |
| Accelerated Phase: — | This is a first-in-human, phase I, open-label, non-randomized |
| Standard Phase: 5 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 1 | Drug: Gamitrinib |
| Accelerated Phase: 10 mg | This is a first-in-human, phase I, open-label, non-randomized |
| Standard Phase: 10 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 2 | Drug: Gamitrinib |
| Standard Phase: 20 mg | This is a first-in-human, phase I, open-label, non-randomized |
| Accelerated Phase: 20 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 3 | Drug: Gamitrinib |
| Accelerated Phase: 40 mg | This is a first-in-human, phase I, open-label, non-randomized |
| Standard Phase: 35 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 4 | Drug: Gamitrinib |
| Accelerated Phase: 80 mg | This is a first-in-human, phase I, open-label, non-randomized |
| Standard Phase: 50 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 5 | Drug: Gamitrinib |
| Standard Phase: 65 mg | This is a first-in-human, phase I, open-label, non-randomized |
| Accelerated Phase: 160 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |
| Experimental: Dose 6 | Drug: Gamitrinib |
| Accelerated Phase: 320 mg | This is a first-in-human, phase I, open-label, non-randomized |
| Standard Phase: 85 mg | dose-escalation and dose-expansion study with the primary |
| | objective to determine the safety profile of small molecule, |
| | mitochondrial-targeted Hsp90 inhibitor, Gamitrinib, including |
| | identification of dose-limiting toxicities (DLT) and maximum |
| | tolerated dose (MTD) in patients with advanced cancers. A |
| | secondary objective of the study is to determine the |
| | recommended dose and regimen(s) for a phase II study. This |
| | study is based on preclinical data demonstrating the anticancer |
| | activity, unique mechanism of action and preliminary safety of |
| | Gamitrinib. |

### Example 2-4: Outcome Measures

### Primary Outcome Measures

Determine the MTD and/or RP2D of Gamitrinib when administered once weekly. [Time Frame: 7 years]. First cycle dose-limiting toxicities. The maximally tolerated dose (MTD) is defined as the dose level below which the absolute observed DLT rate is >25%. The MTD is equivalent to the anticipated recommended phase 2 dose (RP2D).

### Secondary Outcome Measures

1. Evaluate the overall safety profile of intravenously administered single-agent Gamitrinib [Time Frame: 7 years]. Overall, safety profile of Gamitrinib as characterized by type, frequency, severity, timing and relationship to study therapy of adverse events and laboratory abnormalities according to NCI CTCAE v5.0.
2. To evaluate the peak concentration (Cₘₐₓ) of Gamitrinib [Time Frame: 7 years]. Blood sample is collected for determination of Gamitrinib plasma concentrations. The PK parameter is evaluated includes peak concentration (Cₘₐₓ).
3. To evaluate the area under the concentration time curve (AUC₀₋ₜ) of Gamitrinib [Time Frame: 7 years]. Blood sample is collected for determination of Gamitrinib plasma concentrations. PK parameter is evaluated includes area under the concentration time curve (AUC₀₋ₜ).
4. To evaluate the clearance (CL) of Gamitrinib [Time Frame: 7 years]. Blood sample is collected for determination of Gamitrinib plasma concentrations. PK parameter is evaluated includes clearance (CL).
5. To evaluate the time to maximum concentration (Tmax) of Gamitrinib [Time Frame: 7 years]. Blood sample is collected for determination of Gamitrinib plasma concentrations. PK parameter is evaluated includes time to maximum concentration (Tmax).
6. To evaluate the coefficient of variation (CV) of Gamitrinib [Time Frame: 7 years]. Blood sample is collected for determination of Gamitrinib plasma concentrations. A coefficient of variation (CV) is assessed at each dose level.
7. Assess the pharmacodynamic effects of Gamitrinib [Time Frame: 7 years].

Assessment of Gamitrinib effects on pharmacodynamic markers. The processed samples are profiled for changes in the expression of 301 metabolites by Ultrahigh Performance Liquid Chromatography/Mass Spectrometry (UPLC/MS) or Gas Chromatography/Mass Spectrometry (G/MS) using the Metabolon, Inc. discovery platform.
8. Document any anti-tumor activity of single agent Gamitrinib [Time Frame: 7 years]. Objective tumor response, as assessed using RECIST 1.1 and RECIL 2017 criteria.

### Example 2-5: Eligibility Criteria

The eligibility criteria of the second study are described in this section, as well as listed in Table 2-4 below.

| | |
|---|---|
| Ages Eligible for Study | 18 Years and older (Adult, Older Adult) |
| Sexes Eligible for Study | All |
| Accepts Healthy Volunteers | No |

### Inclusion Criteria

Histologically confirmed diagnosis of advanced cancer refractory to standard of care therapy, or for whom no standard of care therapy is available. Any numbers of prior therapies are allowed.

Dose escalation phase: Solid tumors and lymphoma may have measurable or evaluable disease as per Response Evaluation Criteria in Solid Tumors (RECIST v. 1.1) or as per RECIL 2017 criteria.

Dose expansion phase: (i). All patients must have at least one site of measurable disease as defined by RECIST v. 1.1. or RECIL 2017, for solid tumors and lymphoma, respectively. (ii). Patients in the expansion cohort must have at least one non-target lesion deemed safe to biopsy, in the opinion of the investigator, and be willing to undergo mandatory core biopsies. This includes pre-treatment and an on-treatment biopsy. Biopsies at the time of progression are highly desired, but optional. (iii). The lesion(s) which is used for response assessment may not be biopsied iv. Target lesions that have been previously irradiated is not considered measurable unless increase in size is observed following completion of radiation therapy.

All previous therapies of cancer, including radiotherapy major surgery and investigational therapies must be discontinued for ≥14 days (≥28 days for mitomycin C or nitrosoureas) before Cycle 1 Day 1 (C1D1), and all acute effects of any prior therapy must have resolved to baseline severity or Grade≤1 Common Terminology Criteria for Adverse Events (CTCAE v5), except alopecia or parameters defined in this eligibility list.

Age>18 years.

ECOG performance status 0-2.

Patients must have normal organ and marrow function as defined below: Absolute neutrophil count ≥1,500/mm³ without growth factor use ≤7 days prior to C1D1 Platelets ≥85,000/mm³ without platelet transfusion ≤7 days prior to C1D1 Hemoglobin >8.5 mg/dL without red blood cell transfusion ≤7 days prior to C1D1 Total serum bilirubin <1.5×upper limit of normal (ULN) (except for patients with documented Gilbert's syndrome) AST (SGOT)/ALT (SGPT)≤2×ULN; ≤5×ULN if liver dysfunction is felt to be secondary to tumor burden Serum creatinine ≤1.5×ULN (OR creatinine clearance ≥60 mL/min/1.73 m²) Serum or urine pregnancy test (WOCBP only) negative ≤7 days of C1D1.

Ability to understand and willingness to sign a written informed consent, HIPAA consent document and comply with the study scheduled visits, treatment plans, laboratory tests and other procedures.

Female patients must be surgically sterile or be postmenopausal, or must agree to use effective contraception during the period of the trial and for at least 90 days after completion of treatment. Male patients must be surgically sterile or must agree to use effective contraception during the period of the trial and for at least 90 days after completion of treatment. The decision of effective contraception is based on the judgment of the principal investigator or a designated associate.

### Exclusion Criteria

Patients with symptomatic brain metastases are excluded. Patients with asymptomatic and treated CNS metastases may participate in this trial. The patient must have completed any prior treatment for CNS metastases >28 days prior to study entry, including radiotherapy or surgery. Concurrent use of steroids for the treatment of brain metastasis are not permitted.

Current treatment on another (therapeutic) clinical trial.

Hypertension not adequately controlled with medications (>150/100 mm Hg despite optimal medical therapy).

Active bacterial fungal or viral infection including hepatitis B (HBV), hepatitis C (HCV), requiring treatment with IV antibiotic, IV anti-fungal, or anti-viral (Testing is not required for eligibility).

Known human immunodeficiency virus (HIV) or acquired immunodeficiency syndrome (AIDS)-related illness (testing is not required for eligibility).

Any of the following in the previous 6 months: myocardial infarction, severe/unstable angina, coronary/peripheral artery bypass graft, symptomatic congestive heart failure, cerebrovascular accident, transient ischemic attack or symptomatic pulmonary embolism.

Other severe acute or chronic medical or psychiatric condition or laboratory abnormality that may increase the risk associated with study participation or study drug administration, or may interfere with the interpretation of study results, or in the judgment of the investigator would make the patient inappropriate for entry into the study

Pregnant or breast feeding.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of treating, ameliorating, or preventing cancer in a subject in need thereof, the method comprising administering to the subject Gamitrinib according to an administration protocol,
wherein the administration protocol comprises a drug dosing phase,
wherein the drug dosing phase comprises a standard dosing phase, during which the amount of Gamitrinib administered weekly to the subject ranges from about 0.1 mg/kg per week to about 50 mg/kg per week.

2. The method of claim 1, wherein the cancer comprises a solid tumor, a lymphoma, or combinations thereof.

3. The method of claim 1, wherein Gamitrinib is the only compound administered to the subject to treat, ameliorate and/or prevent the cancer.

4. The method of claim 1, wherein the drug dosing phase further comprises an acceleration dosing phase, which takes place prior to the standard dosing phase and during which the amount of Gamitrinib administered weekly to the subject is equal to or greater than the amount of Gamitrinib administered weekly to the subject during the standard dosing phase.

5. The method of claim 1, wherein the administration protocol further comprises a recovery phase, which takes place after the dosing phase and during which the subject is not administered Gamitrinib.

6. The method of claim 1, wherein the amount of Gamitrinib administered to the subject during the drug dosing phase is at or below the no observed adverse effect level (NOAEL) or is lower than the highest non-severely toxic dose (HNSTD).

7. The method of claim 1, wherein the maximum serum concentration (Cₘₐₓ) of Gamitrinib during the drug dosing phase is about 2000 ng/mL or less in the subject.

8. The method of claim 1, wherein the subject is further administered with an anti-inflammatory compound and/or a renal protective compound.

9. The method of claim 1, wherein Gamitrinib is formulated as a suspension suitable for parenteral administration.

10. The method of claim 1, wherein the subject is a human.

11. The method of claim 1, wherein the subject is human and the weekly dose in the standard dosing phase comprises about 5 mg/week to about 100 mg/week.

12. The method of claim 1, wherein the subject is human and the weekly dose in the standard dosing phase comprises about 5, 10, 15, 20, 25, 30, 35, 40, 45, 50, 55, 60, 65, 70, 75, 80, 85, 90, 95, or 100 mg/week

13. The method of claim 4, wherein the subject is human and the weekly dose in the acceleration dosing phase comprises about 10 mg/week to about 400 mg/week.

14. The method of claim 4, wherein the subject is human and the weekly dose in the acceleration dosing phase comprises about 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 mg/week.

15. A kit of treating, ameliorating, or preventing cancer in a subject in need thereof, the kit comprising Gamitrinib and an instruction manual, wherein the instruction manual instructs administering to the subject Gamitrinib according to an administration protocol comprising a drug dosing phase, wherein the drug dosing phase comprises a standard dosing phase, during which the amount of Gamitrinib administered weekly to the subject ranges from about 0.1 mg/kg per week to about 50 mg/kg per week.

16. The kit of claim 15, wherein the cancer comprises a solid tumor, a lymphoma, or combinations thereof.

17. The kit of claim 15, wherein Gamitrinib is the only compound in the kit that treats, ameliorates and/or prevents the cancer.

18. The kit of claim 15, wherein the drug dosing phase further comprises an acceleration dosing phase, which takes place prior to the standard dosing phase and during which the amount of Gamitrinib administered weekly to the subject is equal to or greater than the amount of Gamitrinib administered weekly to the subject during the standard dosing phase.

19. The kit of claim 15, wherein the administration scheme further comprises a recovery phase, which takes place after the dosing phase and during which the subject is not administered Gamitrinib.

20. The kit of claim 15, wherein the amount of Gamitrinib administered to the subject during the dosing phase is at or below the no observed adverse effect level (NOAEL), or is lower than the highest non-severely toxic dose (HNSTD).

21. The kit of claim 15, wherein the maximum serum concentration (Cₘₐₓ) of Gamitrinib during the dosing phase is about 2000 ng/mL or less in the subject.

22. The kit of claim 15, wherein the kit further comprises an anti-inflammatory compound and/or a renal protective compound, and the instruction manual instruct administering to the subject the anti-inflammatory compound and/or the renal protective compound.

23. The kit of claim 15, wherein Gamitrinib is formulated as a suspension suitable for parenteral administration.

24. The kit of claim 15, wherein the subject is a human.

25. A pharmaceutical formulation suitable for parenteral formulation, the formulation comprising Gamitrinib and at least one of the following: water, DMSO, polysorbate 80, lecithin (Lipoid S100), sucrose, and dextrose.

26. The pharmaceutical formulation of claim 25, which comprises Gamitrinib, water, DMSO, polysorbate 80, lecithin (Lipoid S100), sucrose, and dextrose.

27. The pharmaceutical composition of claim 25, which comprises:
about 1-25 mg/mL Gamitrinib,
about 0.5-15% DMSO,
about 0.025-1% polysorbate 80,
about 0.01-1% lecithin,
about 0.5-10% sucrose, and
about 1-15% dextrose.

28. The pharmaceutical composition of claim 25, which comprises:
about 5 mg/mL Gamitrinib,
about 2.5% DMSO,
about 0.125% polysorbate 80,
about 0.031% lecithin,
about 1.25% sucrose, and
about 4.375% dextrose.

29. The pharmaceutical composition of claim 25, which is a solution of a suspension.

30. The pharmaceutical composition of claim 25, which is a suspension of average particle size of about 50-500 nm.

31. The pharmaceutical composition of claim 25, which is a suspension of average particle size of about 154 nm.

32. The pharmaceutical composition claim 25, which is a suspension of D(0.9) size of about 50-1000 nm.

33. The pharmaceutical composition of claim 25, which is a suspension of D(0.9) size of about 229 nm.

34. A method of generating a Gamitrinib pharmaceutical formulation suitable for parenteral formulation, the method comprising subjecting to microfluidization a mixture of a DMSO-containing solution of Gamitrinib and a water-containing solution of polysorbate 80, lecithin (Lipoid S100), sucrose, and dextrose.

35. The method of claim 34, which produces a suspension of average particle size of about 50-500 nm.

36. The method of claim 34, which produces a suspension of average particle size of about 154 nm.

37. The method of claim 34, which produces a suspension of D(0.9) size of about 50-1000 nm.

38. The method of claim 34, which produces a suspension of D(0.9) size of about 229 nm.
